(19) United States
(12) Patent Application Publication
(10) Pub. No.: US 2009/0238862 A1
(43) Pub. Date: Sep. 24, 2009

(54) Methods and Compositions for Seprase Inactivation

(76) Inventor: Wen-Tien Chen, Stony Brook, NY (US)

Correspondence Address: PILLSBURY WINTHROP SHAW PITTMAN, LLP, P.O. BOX 10500, MCLEAN, VA, 22102, US

(21) Appl. No.: 11/718,222

(22) PCT Filed: Oct. 27, 2005
(86) PCT No.: PCT/US05/38663

§ 371 Date: May 26, 2009

Related U.S. Application Data
(60) Provisional application No. 60/622,571, filed on Oct. 27, 2004.

(51) Int. Cl. A61K 9/127; C12N 15/11; C12N 15/00; C12N 5/06; A61K 31/7088

(52) U.S. Cl. 424/450; 536/23.1; 435/320.1; 435/325; 514/44 R

(57) ABSTRACT

The present invention relates to isolated nucleic acids encoding short hairpin RNAs that interfere with seprase mRNA expression, vector and host cells for expressing seprase mRNA interfering short hairpin RNAs, and methods of therapeutic use of the same for preventing further tumor invasation and metastasis.

### CROSS-REFERENCE TO RELATED APPLICATIONS

Priority is claimed to U.S. Prov. Appl. No. 60/622,571 filed Oct. 27, 2004.

### BACKGROUND OF THE INVENTION

1. Field of the Invention

The present invention is related to novel nucleic acids, methods and molecular tools with respect to the inhibition of seprase and for the treatment of malignant disorders. More particularly, the present invention relates to isolated nucleic acids encoding short hairpin RNAs that interfere with seprase mRNA expression, vector and host cells for expressing seprase mRNA interfering short hairpin RNAs, and methods of therapeutic use of the same for preventing further tumor invasation and entry into the circulation.

2. Description of Related Art

Metastasis involves multiple steps, in which entry of tumor cells into the circulation (intravasation) is essential. The ability of a tumor cell to intravasate requires that the expression of a specific set of proteases and cell adhesion molecules. Seprase is a 170-kDa type II transmembrane serine protease glycoprotein with a non-classical serine catalytic site that, when dimerized, exhibits dual prolyl dipeptidase and gelatinolytic activities. Seprase and its associated proteolytic activity have been noted in tumor cells and stromal fibroblasts in human melanoma, and invasive breast carcinoma, gastric carcinoma, colonic carcinoma, and cervical carcinoma. It has been shown that seprase is absent or undetectable in all normal tissue cells except in the early stage of wound healing. Seprase was shown to localize at invadopodia protrusions of invasive cells to form hetero-oligomeric complex with homologous dipeptidyl peptidase IV (DPP4/CD26) resulting in degradation of the extracellular matrix (ECM) in contact with the cell; further association of the protease complex with α3β1 integrin promotes the cell to form adhesion contact with the extracellular matrix (ECM). Importantly, seprase has been shown to be involve in promoting tumor growth in animal models.

Nothwithstanding interest in seprease as a potential target for cancer therapy, therapies that employ novel gene targeted and immuno targets against seprase have not been described. The present provides novel nucleic acids, methods and molecular tools with respect to the inhibition of seprase and for the treatment of malignant disorders.

### SUMMARY OF THE INVENTION

The present invention is related to applications of interfering RNA (RNAi) vectors in metastasis model studies that provide powerful tools for molecular analysis of the metastatic process as well as identification of genes controlling tumor intravasation. This invention is also related to therapeutic methods that target seprase in order to localize and suppress tumors at their primary sites relying on seprase's lack of expression in normal tissues vs. its high expression in tumor cells and tumors.

The present invention relates to an isolated nucleic acid molecule encoding short hairpin RNAs (shRNAs) specific to seprase messenger RNA (mRNAs) to form RNA/protein complexes (RISC) that are recognized by Dicer proteins that destroy seprase mRNAs thereby preventing translation of seprase mRNA. The isolated nucleic acid molecule comprises a nucleotide sequence selected from the group consisting of the nucleotide sequence as set forth in SEQ ID NO: 3, the nucleotide sequence as set forth in SEQ ID NO:4, and a nucleotide sequence fully complimentary to SEQ ID NO: 3 or SEQ ID NO: 4. The present invention also provides a vector comprising the nucleic acid molecule encoding shRNAs that target seprase mRNAs with a heterologous promoter DNA that is operatively linked to these nucleotide sequences. The present invention additionally provides for a host cell that comprises the vector of the invention. The present invention also relates for liposomes comprising the vectors comprising the nucleotide sequences encoding shRNAs against seprase mRNAs wherein the liposomes may contain tumor homing molecules to target the liposomes to tumor cells.

The present invention also further relates to a vector for suppressing seprase mRNA translation comprising a promoter operable in a mammalian host cell, an oligonucleotide sense sequence that targets a seprase mRNA gene sequence, an oligonucleotide spacer sequence, and an oligonucleotide anti-sense sequence of the same seprase mRNA gene sequence. This vector is capable of using either an inducible or constitutively driven promoter or promoter that is recognized in a tumor cell by the tumor cell machinery (e.g., the U6 promoter). Oligonucleotide sequences specific to particular regions of the human or mammalian seprase gene may be provided with the vector and inserted in a sense, spacer and antisense orientation. The present invention is also directed to host cells and liposomes harboring the vector as described above.

The present invention also provides for a method for inhibiting tumor intravasation by administering a therapeutically effective amount of a vector in a patient in need thereof wherein the vector comprises nucleotide sequences that reduce native seprase mRNA translation in tumor cells by forming a short hairpin inhibitory RNA that targets and forms complexes that destroy native seprase mRNAs. The present invention further relates to decreasing the overall seprase activity in tumor cells, prevents the formation of the hetero-oligometric protease complex comprising seprase and dipeptidyl peptidases (DPP4/CD26) and preventing the interaction between seprase and α3β1 intergrin. The method of the present invention employs the vector that contains polynucleotide sequence encoding the shRNAs that target seprase mRNAs and may effectively target tumor cells such as, but not limited to, melanoma, breast cancer cells, gastric carcinoma cells, lung, liver, colonic carcinoma cells, and cervical carcinoma cells.

The present invention also provides for seprase polypeptides and it biologically active fragments and variants thereof that maybe used for therapeutic or diagnostic purposes to localize and suppress malignancies or tumors at their primary site. Representative seprase polypeptides of the invention include isolated polypeptides comprising the amino acid sequence selected from the group consisting of the amino acid sequence for the 90 kDa seprase subunit as set forth in SEQ ID NO: 27; the amino acid sequence for the 35 kDa truncated seprase subunit as set forth SEQ ID NO: 22; the amino acid sequence for the 25 kDa truncated seprase subunit as set forth in SEQ ID NO: 23; and the amino acid sequence for a seprase recombinant fragment of the native seprase as set forth in SEQ ID NO 26. The present invention provides methods for using the seprase polypeptides and their biologically active fragments and variants therein for therapeutic purposes to localize and suppress tumor growth and metastasis.

The present invention also provides for murine monoclonal anti-seprase antibodies and antibody fragments, and method for preparing and using the same. The anti-seprase antibodies mAb 65, mAb 68, mAb 82, and mAb 90 comprise at least one light chain or at least one heavy chain, or fragments thereof, wherein the anti-seprase antibody or antibody fragment (a) specifically binds to human seprase antigen with a binding affinity of at least about 1×10⁻⁷ M to about 1×10⁻¹² M; (b) specifically binds to human seprase antigen with a binding affinity greater than 1×10⁻¹¹ M; (c) specifically binds to human seprase antigen with a binding affinity greater than 5×10⁻¹¹ M; (d) specifically targets seprase-expressing cells in vivo; (e) competes for binding to human seprase with an antibody of any one of (a)-(d); (f) specifically binds to an epitope bound by any one of (a)-(d); or (g) comprises an antigen binding domain of any one of (a)-(d). The murine anti-seprase antibodies mAb 65, mAb 68, mAb 82, and mAB90 of the invention comprise constant regions that are derived from human constant regions, such as IgG1 or IgG4 constant regions.

The present invention also provides for chimer and humanized anti-seprase antibodies and antibody fragments, and method for preparing and using the same. The anti-seprase antibodies of the invention comprise at least one light chain or at least one heavy chain, or fragments thereof, wherein the chimeric or humanized anti-seprase antibody or antibody fragment (a) specifically binds to human seprase antigen with a binding affinity of at least about 1×10⁻⁷ M to about 1×10⁻¹² M; (b) specifically binds to human seprase antigen with a binding affinity greater than 1×10⁻¹¹ M; (c) specifically binds to human seprase antigen with a binding affinity greater than 5×10⁻¹¹ M; (d) specifically binds to human seprase antigen with a binding affinity greater that a binding affinity of murine anti-seprase antibody binding to human seprase antigen; (e) specifically targets seprase-expressing cells in vivo; (f) competes for binding to human seprase with an antibody of any one of (a)-(e); (g) specifically binds to an epitope bound by any one of (a)-(e); or (h) comprises an antigen binding domain of any one of (a)-(e). Chimeric and humanized anti-seprase antibodies of the invention comprise constant regions that are derived from human constant regions, such as IgG1 or IgG4 constant regions. The anti-seprases antibodies of the present invention may also be used therapeutic applications to treat and/or prevent tumor growth and metastasis.

### BRIEF DESCRIPTION OF THE DRAWINGS

FIG. 1. Coupled GFP expression and Seprase suppression in human melanoma cells.

(a) pGUS vector. The human U6 promoter sequence was amplified from plasmid pGEM/U6. pGUS was constructed by directionally inserting the U6 sequence into BspLU 11I/Ase I digested plasmid pEGFP-C1.

(b) RNAi vectors expressing shRNAs. Two oligonucleotides containing a 20-nt sense and an 20-nt antisense sequence of target mRNA, a 7-nt spacer sequence, and a U6 transcription termination signal of 6 thymidines were annealed and inserted into pGUS between the unique Bpu AI and BspLU 11I sites, immediately downstream of the U6 promoter. Synthetic oligonucleotides for generation of vectors targeting seprase (PGUS-SEP1384 and PGUS-SEP1821) and a vector targeting no human gene (pGUS-NO, RNAi negative control vector) are depicted.

(c) Real-time RT-PCR analysis of seprase mRNA expression in parental LOX sublines (LOX-1 and LOX-2), pGUS transfected GUS sublines (GUS-1), pNO transfected NO sublines (NO-1) and pGUS-SEP transfected SEP sublines (SEP-1 and SEP-2). Seprase versus β-actin mRNA ratio derived from LOX-1 cells was normalized to 100.

(d) RNAi suppression of seprase protein expression in LOX sublines. Western immunoblotting analysis was performed to compare the protein expression levels of seprase (arrow), MT1-MMP and actin in stable LOX sublines by using mouse mAb 90 against seprase dimers, rabbit antibody against MT1-MMP hinge region and mAb anti-actin clone AC-40, respectively. Purified recombinant seprase protein (r-sep) was used as a control.

(e) Proteolytic activities specific for seprase. The prolyl dipeptidase (Gly-Pro-pNA cleavage) and serine gelatinase (DQ gelatin degradation) activities of seprase were measured using immuno-capture proteolytic assays for cell lysate of stable LOX sublines. The right two bars indicate negative and positive controls: PBS and recombinant seprase (r-sep).

(f) DNA microarray to compare the gene expression profiles of seprase and interferon target genes in LOX sublines. Data correspond to the top 9 RNAi interferon target genes (Bridge et al., Nat. Genet. 34:263-4 (2003) that are represented by 10 probe sets in the Affymetrix DNA microarray Hu133A GeneChip. Each column represents one LOX subline; each row one gene or probe set. Different levels of gene expression are represented on a scale from dark blue (lowest expression) to dark red (highest expression). The Affymetrix probe set code, p value and fold change are shown. Genes are ranked by descending p values; * indicates the P Call value cross the six sublines that is greater than 50%. Note that the expression of seprase mRNA is uniformly suppressed in SEP sublines whereas the expression of interferon target genes in the two cell groups is not significantly changed (p>0.05).

(g) Expression pattern of seprase and MMP genes. Data correspond to 22 MMP genes that are represented by 37 probe sets in the Hu133A GeneChip. Note that the expression change of the MMP genes in these two cell groups is not significant (p>0.05).

(h) Expression pattern of seprase and related serine protease genes. Data correspond to 30 serine protease genes that are represented by 34 probe sets in the Hu133A GeneChip. Comparison of serine protease gene expression between GUS and SEP cell groups indicates that the change of expression of the 30 serine protease genes in these two cell groups is not significant (p>0.05). Note that the second top protease (tryptase gamma 1) has a p-value of 0.02 but its P Call is 0, suggesting a questionable association. Serine protease genes were referred to a serine protease collection (Netzel-Arnett et al., Cancer Metastasis Rev. 22:237-58 (2003) and MEROPS Protease Database.

(i) Genes associated with seprase expression in LOX cells. Hierarchical cluster diagrams of top 18 genes up-regulated in GUS sublines and top 10 genes up-regulated in SEP sublines are shown. These differentially regulated genes were selected based on three standards: 1) P Call≧50%, 2) p value≦0.05; and 3) fold change≧2. Data were displayed in a manner similar to that shown in a above, except that genes are ranked by descending fold change values.

FIG. 2. Cells with high seprase expression exhibit the invasive phenotype in culture.

(a) Shape of cells with altered seprase expression. No difference was detected in cells grown on plain plastic surface. Bar=100 μm.

(b) Cells cultured in 3D Matrigel. No difference was detected in cells grown in 3D Matrigel according to the method described (Weaver et al., J. Cell Biol. 137:231-45 (1997). Bar=100 μm.

(c) Proliferation of cells cultured on 2D and in 3D Matrigel. Cells were seeded at an initial density of 5×10³ cells/well atop or within Matrigel gels and cultured for 10 days according to a method described (Hotary et al., Cell 114:33-45 (2003). After dissolving gels in 1 mg/ml Collagenase/Dispase (Roche) solution, cell number was determined by hemacytometry.

(d) Proliferation of cells cultured on 2D and in 3D soft agar. Cells were seeded at an initial density of 5×10³ cells/well atop a thin layer of agarose gel or embedded in agarose gel, and cultured for 8 and 14 days, respectively. The number of cells atop agarose gel was determined by hemacytometry; while the number of soft agar colonies within the gels was counted in ten microscopic fields according to a method described (Yang et al., Clin. Cancer Res. 5:3549-3559 (1999).

(e) Cell invasion phenotype in culture as determined by local removal of fibronectin-coated gelatin film by the cell. LOX sublines were cultured on FITC-fibronectin-coated crosslinked gelatin films as previously described (Chen et al., J. Tiss. Cult. Meth. 16:177-81 (1994). Cells were fixed with 20% formaldehyde to quench GFP fluorescence in order to visualize FITC labeled fibronectin-gelatin films. The fixed cells and FITC-fibronectin-gelatin films were then photographed using differential interference contrast microscopy for cell morphology (upper panels) and epifluorescence microscopy for cell invasiveness (lower panels). The lower panels show the appearance of dark spots in the fibronectin-gelatin films underlying GUS-1 and NO-1 cells, and reduction of dark spots under seprase-suppressed SEP-1 and SEP-2 cells. Bar=50 μm.

(f) Degradation of type I collagen/gelatin by cells embedded in 3D type I collagen gel. 1×10⁵ cells per well were embedded within TRITC-collagen type I gels, incubated and measured according to a method described (Ghersi et al., J. Biol. Chem. 277:29231-41 (2002). Release of TRITC-peptides from the gel indicates degradation of collagen/gelatin by cells. Control is reading from parallel wells containing no cell.

(g) Formation of vasculogenic-like networks of cells cultured in 3D Matrigel. Cells were seeded on top of the gel at 1×10⁴ cells per well and incubated for 10 days according to a method described (Maniotis et al., Am. J. Pathol. 155:739-52 (1999).

FIG. 3. Cells with high seprase expression generate rapidly growing tumors with vasculogenic mimicry.

(a) Growth of primary tumors in SCID mice that were inoculated s.c. with LOX sublines.

(b) Proteolytic activities specific for seprase in the tumors. Tumors derived from LOX-1, GUS-1 and SEP-1 cells were lysed with 1% octyl glucoside, 5 mM EDTA in PBS. Protein enriched by WGA affinity chromatography was then subjected to immuno-capture proteolytic assays to measure prolyl dipeptidase (Gly-Pro-pNA cleavage) and serine gelatinase (DQ gelatin degradation) activities of seprase. PBS and r-seprase were used as controls.

(c) Seprase protein expression in the tumors. Tumors were lysed with 1% octyl glucoside, 5 mM EDTA in PBS. Tumor lysate was first immunoprecipitated using mAbs D8, D28 and D43 (against seprase) conjugated beads, followed by Western immunoblotting using mAb E97. R-sep, recombinant seprase; C, control, rat IgGs eluted from mAbs D8, D28 and D43 conjugated beads; bands marked by an arrow are seprase subunits.

(d) The 170-kDa gelatinolytic activity present in the GUS tumor. Tumor lysate was first enriched by WGA affinity chromatography; WGA binding protein with (+) or without (−) prior depletion by mAbs D8, D28 and D43 conjugated beads was then subjected to gelatin zymography. Note that the gelatinolytic activity specific for seprase (arrow) is only detectable in GUS tumors, and it appears to be mainly human form of seprase (derived from human tumor cells).

(e and f) Histological sections of tumors derived from GUS-1 (e) and SEP-1 (f) cells. Sections were stained with hematoxylin and eosin. Note that GUS tumors show more vessel-infiltrating growth and larger areas of red blood cell clusters. Bar=100 μm.

(g) Percentage of red cell clusters in a given area of tumor sections. Average number of red cell clusters in an area of 331 μm×239 μm in GUS-1 tumor sections (n=10) is normalized to 100%.

(h and i) Enlarged histological views of GUS and SEP tumors. Note that red blood cell clusters are enclosed by tumor cells without the presence of an endothelial lining. Bar=100 μm.

(j) Number of microvessels in GUS and SEP tumors. Blood vessels in tumor sections were revealed by staining with anti-von Willebrand Factor antibody. Blood vessels were counted in a given area (331 μm×239 μm) of tumor sections (n=10).

(k) Number of tumor/stromal cells and percentage of proliferating cells in GUS and SEP tumors. Tumor and stromal cells and proliferating (Ki67 positive) cells were counted in a given area (331 μm×239 μm) of tumor sections (n=10).

(l) Number of apoptotic cells in GUS and SEP tumors. Apoptotic cells were revealed by staining with Cleaved Caspase-3 (Asp175) antibody, and they were counted in a given area (331 μm×239 μm) of tumor sections (n=10).

FIG. 4. Cells with high seprase expression produce circulating tumor cells and micrometastases.

(a) Circulating tumor cells isolated from SCID mice that were inoculated s.c. with cells with contrasting levels of seprase expression. Bar=100 μm.

(b) Number of circulating tumor cells in a SCID mouse inoculated s.c. with tumor cells. Cell number was estimated by counting colonies in culture derived from approximately 2-mL of blood per mouse.

(c) Number of circulating tumor cells in a SCID mouse inoculated s.c. with mixed types of tumor cells. Cell number was estimated by counting colonies in culture derived from approximately 2-mL of blood per mouse. SCID mice were inoculated s.c. with mixed LOX-1 and GUS-1 cells (LOX+GUS) or LOX-1 and SEP-1 cells (LOX+SEP). LOX˜SEP indicates control mice that were injected with LOX-1 cells on one side and SEP-1 cells on the other side of mouse. Note that SEP-1 cells intravasate when SEP-1 cells were co-injected with LOX-1 cells (LOX+SEP) but not when separately injected (LOX˜SEP) into the same mouse.

(d) Lung and liver micrometastases developed in SCID mice that were inoculated s.c. with cells with contrasting levels of seprase expression. Bar=100 μm.

(e) Number of lung and liver micrometastases in SCID mice inoculated s.c. with tumor cells. Number of micrometastases in lung and liver was estimated by counting GFP-labeled micrometastases under an epifluorescence microscope at the time mice were sacrificed.

(f) Lung and liver micrometastases in a SCID mouse inoculated s.c. with mixed types of tumor cells. Bar=100 μm.

(g) Number of lung and liver micrometastases in a SCID mouse inoculated s.c. with mixed types of tumor cells. Note that SEP-1 cells metastasize to lung and liver when SEP-1 cells were co-injected with LOX-1 cells (LOX+SEP) but not when separately injected (LOX˜SEP) into the same mouse.

FIG. 5. Role of seprase in growth of metastatic colonies.

(a) Lung and liver macrometastases developed in SCID mice that were inoculated s.c. with cells with contrasting levels of seprase expression and had their primary tumors removed at day 20. Lung and liver metastases were viewed by epifluorescence microscopy. Bar=100 μm.

(b) Number of lung and liver macrometastases in SCID mice inoculated s.c. with tumor cells with contrasting levels of seprase expression and had their primary tumors removed at day 20.

(c) Lung metastatic colonies in SCID mice that were injected i.v. with cells with contrasting levels of seprase expression. Bar=100 μm.

(d) Number and size of lung macrometastases in SCID mice that were injected i.v. with cells with contrasting levels of seprase expression.

FIG. 6. Seprase Promotes Fibrosarcoma Cells Intravasation and Tumor Growth

(a) Seprase over-expression vector pE15. The cDNA regions encoding seprase predicted domains are indicated. C, seprase cytoplasmic domain, amino acid 1-6; TM, transmembrane domain, amino acid 7-26; S, stalk region, amino acid 27-48; GR, glycosylation rich region, amino acid 49-314; CR, cysteine rich domain, amino acid 305-466; CAT, catalytic domain, amino acid 493-760. The cDNA fragment encoding seprase extracellular domain was cloned into a CMV promoter expression cassette on plasmid pE0 in frame with an N-terminal mouse Igk secretion signal and a C-terminal V5-His tag.

(b) Stable HT1080 sublines doubly transfected with pGUS+pE0 (HT-0) or pGUS+pE15 (HT-15). Cells were photographed using phase contrast microscopy for cell morphology (upper panels) and epifluorescence microscopy for GFP (lower panels). Bar=100 μm.

(c) Over-expression of seprase. Cell culture medium conditioned by HT-0 and HT-15 cells was concentrated 10-fold, and then analyzed by Western immunoblotting. Culture media of 293-EBNA cells transfected with pE0 and pE15 were used as controls. Note that HT-15 cells produced more seprase protein (arrow) than HT-0 cells.

(d) Proteolytic activities specific for seprase. The prolyl dipeptidase (Gly-Pro-pNA cleavage) and serine gelatinase (DQ gelatin degradation) activities of seprase were measured using immuno-capture proteolytic assays for media conditioned by HT-0 and HT-15 cells.

(e) Tumors derived from HT-0 and HT-15 cells in the skin of SCID mice.

(f) Lung micrometastases in SCID mice that were inoculated s.c. with HT-0 and HT-15 cells. Note that HT-15 subline produced more lung micrometastases than HT-0 subline. Bar=100 μm.

(g) Quantification of lung micrometastases. Number of micrometastases was estimated by converting GFP-cell counts per unit of area measured by epifluorescence microscopy.

(h) Proteolytic activities specific for seprase in tumors. Tumors derived from HT-0 and HT-15 cells were lysed with 1% octyl glucoside, 5 mM EDTA in PBS. Total protein enriched by WGA affinity chromatography from tumor lysate was subjected to immuno-capture proteolytic assays to measure prolyl dipeptidase (Gly-Pro-pNA cleavage) and serine gelatinase (DQ gelatin degradation) activities of seprase. PBS and r-seprase were used as controls.

(i) Protein specific for human seprase in tumors. Total protein in tumor lysate was enriched by WGA affinity chromatography and then subjected to Western immunoblotting with (+) or without (−) prior depletion by immuno-absorbent beads conjugated with mAbs D8, D28 and D43. Seprase revealed by mAb E97 are indicated by an arrow.

(j) Lung metastatic colonies in SCID that were derived from i.v. inoculation of HT-0 and HT-15 cells in 20 days after cell injection. Bar=100 μm.

(k) Quantification of number and size of lung metastatic colonies in SCID that were derived from i.v. inoculation of HT-0 and HT-15 cells.

FIG. 7. Identification of s-seprase in human tumors.

(a-b) Positive antibody staining (panel a) and negative control staining (panel b) with hematoxylin for seprase in melanoma cells (open arrow) and stromal cells (arrow) of a malignant melanoma tissue using mAb D8 directed against seprase. Bar=50 μm.

(c-f) Parallel SDS PAGE analyses on s-seprase in tumors (T) and adjacent normal tissues (N) by gelatin zymography and immunoblotting using mAbs D8 and E97. WGA-binding proteins purified from paired tumor (T) and adjacent normal (N) tissues from same patients were analyzed in human malignant melanoma (panel c), breast carcinoma (panel d), colon carcinoma (panel e) and gastric carcinoma (panel f) in parallel by gelatin zymography and immunoblotting. Gelatinolytic activities and proteins specific for seprase were determined in the conditions to resolve all types of gelatinases (AG) and the incubation of gelatin zymograms in the presence of 5 mM EDTA to resolve serine-type gelatinase activities (SG). A major gelatinase activity of the protein at 70 kDa is prominent and specific in all tumor tissues.

(g) N-seprase in LOX cell lines that stably express seprase at differential levels. LOX cells with low seprase expression were generated by RNAi knockdown using the vector pGUS-SEP1384, followed by stable cell selection, and cells with high seprase was initiated used control pGUS vector. Cell lysates of LOX (sep^{high}) and LOX (sep^{low}) were compared to r-seprase by Western immunoblotting using mouse mAb 90 (against r-seprase) and rat mAb E97 (against denatured n-seprase), and anti-actin mAb clone AC-40. Protein samples were boiled for 3 min before Western immunoblotting using mAb E97 and anti-actin mAb clone AC-40. Lanes labeled r-sep are r-seprase as a positive control; the band labeled Anti-actin serves as a protein loading control.

(h) The subunit composition of s-seprase in human tumors. Tumor tissue lysates prepared from experimental tumors derived from LOX [sep^{high}] cells and LOX [sep^{low}] cells and from human ovarian cancer tissues (patient number OC267, OC268, OC242, OC244, OC261) were immunoprecipitated using the mAbs D8-D28-D43 conjugated beads. The antigen-antibody complexes were solubilized in SDS sampling buffer and subjected to SDS PAGE and Western immunoblotting using mAb E97. R-seprase (r-sep) was used as a positive control. The mAb D8-D28-D43 beads without mixing with tumor lysate were used as control for mAbs that were conjugated on the beads, which showed heavy and light chains of rat mAb IgG (▪), heavy chain alone () and light chain alone (▪). The bands indicated by arrows are various forms of seprase present in the tumors.

(i) Dipeptidyl peptidase (DP) and gelatinase activities specific for seprase in the immuno-affinity purified protein from various tumors. Samples were prepared similarly as in (panel h) above. The soluble enzymatic assays were performed in the presence of 5 mM EDTA to suppress potential MMP activity. PBS was used as a negative control. Three experiments for each condition were performed. The values are mean±SD. Significance symbol * p<0.05 above bar is used to confirm the specific activity of seprase.

FIG. 8. Isolation and characterization of r-seprase.

(a) The cDNA regions encoding predicted domains of seprase are indicated: C, cytoplasmic domain, amino acid 1-6; TM, transmembrane domain, amino acid 7-26; S, stalk region, amino acid 27-48; GR, glycosylation rich region, amino acid 49-314; CR, cysteine rich domain, amino acid 305-466; CAT, catalytic domain, amino acid 500-760. The cDNA fragment encoding r-seprase lacks the cytoplasmic and transmembrane domains and was inserted into a CMV promoter driven expression cassette containing an N-terminal mouse Igk secretion signal and a C-terminal V5-His tag.

(b) A gelatin zymogram (left panel) and a corresponding Western immunoblot (right panel) of r-seprase. The culture medium conditioned by the pE15 transfected 293-EBNA cells were prepared under non-boiling (−) or boiled (+) conditions, and subsequently resolved on a gelatin zymogram and a Western immunoblot using anti-V5 antibody. The gelatinase activity of r-seprase was detected by incubating the zymogram in the presence of 5 mM EDTA.

(c) Dissociation of the active 160 kDa r-seprase into the inactive 90 kDa monomer on a gelatin zymogram. Freshly purified r-seprase is active at 160 kDa (fresh); r-seprase stored at −20° C. is moderately active as a dimer and a portion is dissociated into a 90 kDa inactive monomer (stored); r-seprase stored at −20° C. and then incubated at 37° C. overnight is also active as a dimer and a portion is dissociated into a 90 kDa inactive monomer (incubated); the purified protein that was boiled for 3 min is dissociated into a 90 kDa inactive monomer (boiled). Note that r-seprase dimers show white bands against the blue background due to their gelatinase activity, whereas the monomers show dark blue bands compared to the gelatin background.

(d) Comparison of the gelatinase activity of n-seprase and r-seprase by parallel gelatin zymography and Western immunoblotting analyses. LOX cell detergent lysate (n-sep) and the culture medium conditioned by the pE15 transfected 293-EBNA cells (r-sep) were WGA-affinity purified. Resulting protein complexes were subjected to gelatin zymography (left panel) for gelatinase detection and Western immunoblotting using mAb 90 (right panel) for assessment of relative amounts of seprase.

(e) The proteolytic activities of r-seprase. The DP and gelatinase activities specific for seprase were measured using the soluble enzymatic assays on immunopurified protein (Ghersi et al., J. Biol. Chem. 277:29231-41 (2002). Seprase was isolated from the culture medium conditioned by the pE15 transfected 293-EBNA cells using rat mAbs D8 and D43 (against n-seprase), mAb E97 (against dissociated subunits and polypeptides of n-seprase) and mouse mAbs 90 and 65 (against r-seprase). The DP (Gly-Pro-pNA cleavage) and gelatinase (DQ gelatin degradation) activities of the isolated r-seprase were measured in parallel. PBS was used as a control. Significance symbol * p<0.05 above bars are used to confirm specific activity.

FIG. 9. Proteolytic truncation of seprase activates its gelatinase activity.

(a) Increased gelatinase activity of purified r-seprase incubated at 37° C. R-seprase samples were sequentially isolated by a DEAE Sepharose column, a WGA affinity chromatography column, and a His•Bind Resin column. Purified proteins were incubated at 4° C. (left panel) or 37° C. (right panel) overnight, and then subjected to gelatin zymography. Each lane corresponds to the r-seprase present in 10 mL of original culture medium.

(b) Activation of the 160 kDa r-seprase into the 50 kDa gelatinase. R-seprase was enriched by WGA-affinity chromatography column and incubated at 4° C. or 37° C. for 1 day in the presence or absence of 5 mM EDTA (indicated by 37° C.+EDTA and 37° C.−EDTA, respectively), and then subjected to gelatin zymography and Western immunoblotting using mAb D8.

(c) Activation of r-seprase to increase gelatinase activity but not DP activity. R-seprase was enriched by WGA-affinity chromatography column and incubated at 4° C. or 37° C. for 1 day in the presence or absence of 5 mM EDTA (indicated by 37° C.+EDTA and 37° C.−EDTA, respectively), and then subjected to the soluble enzymatic assays as described by (Ghersi et al., J. Biol. Chem. 277:29231-41 (2002). PBS was used as a negative control in the soluble enzymatic assay. The values are mean±SD. Significance symbol * p<0.05 above bar is used to confirm specific increase in gelatinase activity of truncated seprase.

(d) SDS PAGE analysis on dissociated subunits of purified r-seprase. R-seprase samples indicated were sequentially isolated by the DEAE Sepharose column (DEAE), WGA affinity chromatography column (WGA) and His•Bind Resin column (His) from the culture medium conditioned by the pE15 transfected 293-EBNA cells, followed by heating at 100° C. for 3 min and running SDS PAGE on a 10% gel. The gel was stained with Coomassie Brilliant Blue. Each lane corresponds to the r-seprase present in 10 mL of original culture medium.

(e) Increased gelatinase activity of the r-seprase treated by trypsin. R-seprase isolated by His column was mixed with pure trypsin. The mixture (R-sep+trypsin) and pure enzymes (R-sep and Trypsin) were incubated at 37° C. for 2 h and then subjected to gelatin zymography. Individual purified r-seprase (R-sep) and trypsin (Trypsin) were used as controls.

(f) Increased gelatinase activity but not the DP activity of the r-seprase treated by trypsin. Preparation of the enzyme mixture (R-sep+trypsin) and pure enzymes (R-sep and Trypsin) was identical to these shown in (panel e). The soluble enzymatic assays were performed in the presence of 5 mM EDTA. Three experiments for each condition were performed. The values are mean±SD. Significance symbol * p<0.05 above bar is used to confirm specific increase in gelatinase activity of trypsin-truncated seprase.

FIG. 10. Amino Acid Sequence For Variants of Seprase.

(a) Amino acid sequence for R-seprase.

(b) Amino acid sequence for 35 kDa s-seprase.

(c) Amino acid sequence for 25 kDa s-seprase.

### DETAILED DESCRIPTION OF THE INVENTION

As used herein, seprase (fibroblast activation protein α) refers to the extracellular matrix degrading protein (see U.S. Pat. No. 5,767,242). Seprase or fibroblast activation protein α, when dimerized, exhibits dual prolyl dipeptidase and gelatinolytic activities (Aoyama and Chen, Proc. Natl. Acad. Sci. USA 87:8296-8300 (1990); Goldstein et al., Biochem. Biophys. Acta 1361:11-19 (1997); Park et al., J. Biol. Chem. 274:36505-12 (1999); Smaylan et al., Proc. Natl. Acad. Sci. USA 91:5657-61 (1994).

Interference RNA (RNAi) refers to the process by which double-stranded RNA molecules (dsRNAs) specifically silence the expression of its cognate messenger RNA (mRNA). RNAi constructs may thus be used to diminish and nearly abolish specific gene expression. In the context of the present invention, RNAi constructs function by preventing and/or interfering with the transcription of seprase mRNA, thereby leading to diminished seprase protein production.

RNAi type nucleic acids may be nucleic acids that encode RNA that forms double stranded RNA molecules either encoding 100%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, 0.1%. 0.01%, 0.001%, 0.0001% of the targeted gene such as, but not limited to the mammalian and human seprase gene. These double stranded RNA molecules form airpin RNA loops varying in length depending upon percent of nucleotides targeted of a particular gene of interest such as, but not limited to the mammalian and human seprase gene.

As used herein, dsRNAs refers to any double-stranded RNA molecule. Dicer, a nuclease specific to RNA duplexes, cleaves such dsRNA molecules into small interfering RNA molecules (siRNA). siRNA is a specific types of dsRNA molecule comprising usually of 20-25 ribonucleotides. Another type of dsRNA molecule is short hairpin RNA (shRNA), wherein the sense and antisense RNA sequences of the target gene are connected by a hairpin loop. In the embodiment of the invention, shRNA specific to seprase is used to suppress seprase expression in vivo and in vitro.

microRNA—process by which a single stranded RNA silences gene expression.

RNAi constructs may be used to interfere with expression of endogenous seprase. Specific RNAi constructs harbor or comprise DNA sequences that encode for shRNA sequences for seprase. The shRNA is at least 15, 18, 21, or 24 nucleotides of the complement of the seprase mRNA sequences. The RNAi may have a 2 nucleotide 3′ overhang. If the RNAi is expressed in a cell from a construct, for example, from a polynucleotide encoding a short hairpin molecule or from an inverted repeat of the seprase sequence, then the endogenous cellular machinery will create the overhangs. In addition, RNAi sequences may be prepared by chemical synthesis, in vitro transcription, or digestion of long dsRNA by Rnase III or Dicer. These may be introduced into cells by transfection, electroporation, or other methods known in the art. See, for example, Hannon, Nature 418:244-251 (2002); Bernstein et al., RNA 7:1509-1521 (2002); Hutvagner et al., Curr. Opin. Genet. Dev. 12:225-232 (2002); Brummelkamp, Science 296:550-553 (2002); Lee et al., Nature Biotechnol. 20:500-505 (2002); Miyagishi and Taira, Nature Biotechnol. 20:497-500 (2002); Paddison et al., Genes Dev. 16:948-958 (2002); Paul et al., Nature Biotechnol. 20:505-508 (2002); Sui et al., Proc. Natl. Acad. Sci. USA 99(6):5515-5520 (2002); Yu et al., Proc. Natl. Acad. Sci. USA 99(9):6047-6052 (2002).

RNAi type nucleic acids designed against seprase mRNA may be delivered in vitro to tumor cells or in vivo to tumors. Typical delivery means known in the art may be used. For example, delivery to a tumor may be accomplished by intratumoral injections. Other modes of delivery may be used, including, but not limited to, intravenous, intramuscular, intraperitoneal, intraarterial, and subcutaneous. Conversely in a mouse model, RNAi type nucleic acids may be administered to a tumor cell in vitro, and the tumor cell may be subsequently administered to a mouse.

The polynucleotides of the invention may be DNA or RNA or chimeric mixtures or derivatives or modified versions thereof, single-stranded or double-stranded. The oligonucleotide may be modified at the base moiety, sugar moiety, or phosphate backbone, for example, to improve stability of the molecule, hybridization and the like. The oligonucleotide may include other appended groups such as peptides (e.g., for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA, 84:648-652 (1987); PCT Publication NO: WO88/09810, published Dec. 15, 1988) or the blood-brain barrier (see, e.g., PCT Publication NO: WO89/10134, published Apr. 25, 1988), hybridization-triggered cleavage agents (See, e.g., Krol et al., BioTechniques, 6:958-976 (1988)) or intercalating agents (See, e.g., Zon, Pharm. Res., 5:539-549 (1988)). To this end, the oligonucleotide may be conjugated to another molecule, e.g., a peptide, hybridization triggered cross-linking agent, transport agent, hybridization-triggered cleavage agent,

The oligonucleotide encoding shRNA specific for seprase mRNA may include (but are not limited to) at least one modified base moiety such as 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4 thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, and/or 2,6-diaminopurine.

The oligonucleotide encoding shRNA specific for seprase mRNA may also comprise at least one modified sugar moiety selected from the group including, but not limited to, arabinose, 2-fluoroarabinose, xylulose, and hexose.

In yet another embodiment, the oligonucleotide encoding shRNA specific for seprase mRNA comprises at least one modified phosphate backbone such as (but not limited to), a phosphorothioate, a phosphorodithioate, a phosphoramidothioate, a phosphoramidate, a phosphordiamidate, a methylphosphonate, an alkyl phosphotriester, and a formacetal or analog thereof.

The oligonucleotide encoding shRNA specific for seprase mRNA maybe an a-anomeric oligonucleotide. An a-anomeric oligonucleotide forms specific double-stranded hybrids with complementary RNA in which, contrary to the usual b-units, the strands run parallel to each other (Gautier et al., Nucl. Acids Res., 15:6625-6641 (1987)). The oligonucleotide is a 2-0-methylribonucleotide (Inoue et al., Nucl. Acids Res., 15:6131-6148 (1987)), or a chimeric RNA-DNA analogue (Inoue et al., FEBS Lett. 215:327-330 (1987)).

Polynucleotides of the invention may be synthesized by standard methods known in the art, e.g. by use of an automated DNA synthesizer (such as are commercially available from Biosearch, Applied Biosystems,). As examples, phosphorothioate oligonucleotides may be synthesized by the method of Stein et al. (Nucl. Acids Res., 16:3209 (1988)), methylphosphonate oligonucleotides may be prepared by use of controlled pore glass polymer supports (Sarin et al., Proc. Natl. Acad. Sci. USA, 85:7448-7451 (1988)),

The polynucleotides of the present invention may be employed to inhibit the cell growth and proliferation effects of seprase on neoplastic cells and tissues, i.e. stimulation of angiogenesis of tumors, and, therefore, retard or prevent abnormal cellular growth and proliferation, for example, in tumor formation or growth.

The polynucleotides of the present invention may also be employed to prevent hyper-vascular diseases, and prevent the proliferation of epithelial lens cells after extracapsular cataract surgery. The polynucleotides of the present invention may also be employed to prevent the growth of scar tissue during wound healing. The polynucleotides of the present invention may also be employed to treat, prevent, and/or diagnose the diseases described herein.

The present invention also relates to vectors containing the DNA encoding the seprase shRNAs and host cells by recombinant techniques. The vector may be, for example, a phage, plasmid, viral, or retroviral vector. Retroviral vectors may be replication competent or replication defective. In the latter case, viral propagation generally will occur only in complementing host cells.

The DNA encoding the shRNA against seprase may be joined to a vector containing, a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide of the present invention may be operatively linked to an appropriate promoter, such as the U6 promoter. Other suitable promoters will be known to the skilled artisan. The expression constructs may also contain sites for transcription initiation, termination, and, in the transcribed region, a ribosome binding site for translation.

As indicated, the expression vectors may include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells.

Among vectors for use in the present invention is pGUS. Other suitable vectors will be readily apparent to the skilled artisan.

Introduction of the construct into the host cell may be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). LOX human malignant melanoma cells are the host cells. Appropriate culture mediums and conditions for the above-described host cells are known in the art.

Angiogenesis, also referred to as neovascularization, is defined as the formation of new blood vessels. Angiogenesis is required for normal embryonic development, wound healing, diabetic retinopathy, rheumatoid arthritis, psoriasis, organ regeneration and female reproductive processes. Under these normal physiological processes, angiogenesis is stringently regulated and spatially and temporally delimited.

Angiogenesis is also critical during tumor formation. Without the formation of new blood vessels, tumor cells would be deprived of nutrients and oxygen and therefore be would be unable to grow and metastasize. Unregulated angiogenesis becomes pathologic and sustains progression of many cancers and non-cancerous diseases. A number of serious diseases are dominated by abnormal neovascularization including solid tumor growth and metastases, arthritis, certain types of eye diseases, disorders, and/or conditions, and psoriasis. [See, e.g., reviews by Moses et al., Biotech. 9:630-634 (1991); Folkman et al., N. Engl. J. Med., 333:1757-1763 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983)].

Current research indicates that the growth of solid tumors is dependent on angiogenesis. Folkman and Klagsbrun, Science 235:442-447 (1987). Recent cancer therapies have focused on preventing or inhibiting angiogenesis so that tumor cells fail to grow and metastasize.

Intravasation is defined as the entry of a foreign matter into a blood vessel. In the context of this invention, the foreign matter is a tumor or cancer cell. The spread of tumor cells from a primary tumor to a site of metastasis formation involves multiple interactions such as invasion and degradation of the extracellular matrix (ECM), angiogenesis, intravasation, exit from the circulation (extravasation) and establishment of secondary growth. Because cancer cells reach distant sites by disseminating through blood or lymphatic circulation, intravasation is a crucial event in secondary cancer formation. The breaking down of normal tissue barriers is accomplished by the elaboration of specific enzymes, such as seprase, MMPs, serine proteases that degrade the proteins of the ECN that make up basement membranes and stromal components of tissues, and may assist in cancer invasion and metastasis.

Inhibition of cell motility and invasion would be useful for the treatment of cancer, and other disorders involving cell motility and invasion including those listed above, as well as for contraception. For example, cancer cell invasion driven by altered interactions between cells and an extracellular matrix (ECM). In the case of epithelial-derived carcinomas, the primary tumour is surrounded by a specialized ECM, the basement membrane. Tissue culture procedures which utilize reconstituted basement membrane matrices have been used to demonstrate that changes in matrix deposition, matrix degradation, cellular attachment to the matrix and migration through the matrix play a role in carcinoma cell invasion.

Metastasis is defined as the secondary cancer formation after migrating from the original tumor site via intravasation and extravasation. Current therapies to prevent cancer metastasis focus on reducing and/or abolishing tumor angiogenesis, intravasation, and extravasation. Metastasis is a remarkable process and one which is still poorly understood. The risk of metastases increases as tumours become larger. The cells must survive tissue invasion, circulation, passage across the capillary wall, and establishment in tissues. The process of tissue penetration appears to be by secretion of enzymes known as metalloproteinases (such as collagenase). The precise location of a metastasis is probably due in part to chance. However, clinical patterns of blood-borne metastasis have been observed. For example, gut cancers spread through the portal venous system to the liver; ovarian cancers seed into the peritoneal space; breast cancer has a tendency to spread to the bones of the axial skeleton; and sarcomas often spread into the lung (Souhami, R. L. and Moxham, J., Textbook of Medicine, Second edition, Churchill Livingstone, New York (1994)). A long term goal in the treatment of cancer is the prevention of the spread of the primary tumour by metastasis and the development of secondary tumours elsewhere in the body.

Pharmaceutically acceptable carrier includes any material that when combined with the invention herein disclosed is non-reactive with the immune systems of the subject. Examples include, but are not limited to, any of the standard pharmaceutical carriers such as a phosphate buffered saline solution, water, emulsions such as oil/water emulsion, and various types of wetting agents. Other carriers may also include sterile solutions, and tablets including coated tablets and capsules.

Typically, such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

In certain embodiments, the polynucleotide constructs encoding shRNAs of seprase are complexed in a liposome preparation or lipid-based delivery systems such as a liposome. The lipid based-delivery system may be used to deliver the invention encoding shRNAs to seprase alone or in combination with other anti-cancer drugs or vectors for gene therapy. In this strategy, the DNA encoding shRNAs of the seprase gene are contained in microscopic lipid droplets or other forms of liposomes as described below. The circulation time of these liposomes may be reduce by targeting the liposomes to various tumor specific antigens of particular solid tumor cancers such as, but not limited to, prostate, lung, breast, ovarian, stomach, pancreas, larynx, central nervous tumors, esophagus, testes, liver, parotid, biliary tract, colon, rectum, cervix, uterus, endometrium, kidney, bladder, thyroid, lung, colorectal, and head and neck tumors. These liposomes may target these particular solid tumor antigens by attaching homing molecules at the surface of the liposome. Homing molecules may be various tumor antigens as described above, and include, but are not limited to, BAGE-1 (Boel et al., Immunity 2:167-75 (1995)), GAGE-1, 2, and 8 (Van den Eynde et al., J. Exp. Med. 182:689-98 (1995)), GAGE 3-7 (De Backer et al., Cancer Res. 59:3157-65 (1999)), GnTV (Guilloux et al., J. Exp Med. 183:1173-83 (1996)), HERV K MeI (Schiavetti et al., Cancer Res. 62:5510-6. (2002)), KM-HN-1 (Monji et al., Clin Cancer Res. 10:6047-57. (2004)), LAGE-1 (Rimoldi et al., J Immunol. 165:7253-61. (2000)), MAGE-A1 (Traversari et al., J Exp Med. 176:1453-7. (1992)), MAGE-A2 (Chaux et al., Eur J Immunol. 31:1910-6. (2001)), MAGE-A3 (Gaugler et al., J Exp Med. 179:921-30. (1994)), MAGE-A4 (Kobayashi et al., Tissue Antigens 62:426-32. (2003)), MAGE-A6 (Zorn et al., Eur J Immunol. 29:602-7 (1999)), MAGE-A10 (Huang et al., J Immunol. 162:6849-54 (1999)), MAGE-A12 (van der Bruggen et al., Eur J Immunol. 24:3038-43 (1994)), MAGE-C2 (Ma et al., (2004)), mucin (Jerome et al., J Immunol. 151:1654-62 (1993)), NA-88 (Morea-Aubry et al., (2000)), NY-ESO-1/LAGE-2 (Jager et al., J Exp Med. 187:265-70 (1998); Chen et al., J Immunol. 165:948-55 (2000), Valmori et al., Cancer Res. 60:4499-506 (2000)), Sp17 (Chiriva-Internati et al., Int J Cancer. 107:863-5 (2003)), SSX-2 (Ayyoub et al., J Immunol. 168(4):1717-22 (2002)), Trp2-Int2 (Lupetti et al., J Exp Med. 188:1005-16 (1998)), or peptide variants thereof in various positions of the above-described tumor specific antigens.

These lipid based delivery or liposomal preparations include (1) modifying the lipid composition of the liposome, (2) altering the method of drug entrapment inside liposomes, and (3) incorporating an acid sensitive formulation. The purpose of making the liposome formulation acid-sensitive is to allow release of the nucleic acid encoding the invention within the solid tumor cells following its internalization via the interaction with homing molecules and the tumor antigens expressed on the tumor cell antigen.

Liposomal preparations for use in the present invention include cationic (positively charged), anionic (negatively charged) and neutral preparations. However, cationic liposomes are particularly because a tight charge complex may be formed between the cationic liposome and the polyanionic nucleic acid. Cationic liposomes have been shown to mediate intracellular delivery of plasmid DNA (Felgner et al., Proc. Natl. Acad. Sci. USA, 84:7413-7416 (1987), which is herein incorporated by reference); mRNA (Malone et al., Proc. Natl. Acad. Sci. USA, 86:6077-6081 (1989), which is herein incorporated by reference); and purified transcription factors (Debs et al, J. Biol. Chem., 265:10189-10192 (1990), which is herein incorporated by reference), in functional form.

Cationic liposomes are readily available. For example, N[1-2,3-dioleyloxy)propyl]-N,N,N-trimethylammonium (DOTMA) liposomes are particularly useful and are available under the trademark LIPOFECTIN®, from GIBCO BRL, Grand Island, N.Y. (See, also, Feigner et al., Proc. Natl. Acad. Sci. USA, 84:7413-7416 (1987), which is herein incorporated by reference). Other commercially available liposomes include TRANSFECTACE™ (DDAB/DOPE) and DOTAP/DOPE (Boehringer).

Other cationic liposomes may be prepared from readily available materials using techniques well known in the art. See, e.g. PCT Publication NO: WO 90/11092 (which is herein incorporated by reference) for a description of the synthesis of DOTAP (1,2-bis(oleoyloxy)-3-(trimethylammonio)propane)liposomes. Preparation of DOTMA liposomes is explained in the literature, see, e.g., Felgner et al., Proc. Natl. Acad. Sci. USA, 84:7413-7417, which is herein incorporated by reference. Similar methods may be used to prepare liposomes from other cationic lipid materials.

Similarly, anionic and neutral liposomes are readily available, such as from Avanti Polar Lipids (Birmingham, Ala.), or may be easily prepared using readily available materials. Such materials include phosphatidyl, choline, cholesterol, phosphatidyl ethanolanine, dioleoylphosphatidyl choline (DOPC), dioleoylphosphatidyl glycerol (DOPG), dioleoylphosphatidyl ethanolamine (DOPE), among others. These materials may also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) may be used in various combinations to make conventional liposomes, with or without the addition of cholesterol. Thus, DOPG/DOPC vesicles may be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles may be prepared without sonication to produce multilamellar vesicles or by extrusion through nucleopore membranes to produce unilamellar vesicles of discrete size. Other methods are known and available to those of skill in the art.

The liposomes may comprise multilamellar vesicles (MLVs), small unilamellar vesicles (SUVs), or large unilamellar vesicles (LUVs), with SUVs being. The various liposome-nucleic acid complexes are prepared using methods well known in the art. See, e.g., Straubinger et al., Meth Immunol 101:512-527 (1983), which is herein incorporated by reference. For example, MLVs containing nucleic acid may be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochem. Biophys. Acta, 394:483 (1975); Wilson et al., Cell 17:77 (1979)); ether injection (Deamer et al., Biochem. Biophys. Acta, 443:629 (1976); Ostro et al., Biochem. Biophys. Res. Commun., 76:836 (1977); Fraley et al., Proc. Natl. Acad. Sci. USA, 76:3348 (1979)); detergent dialysis (Enoch et al., Proc. Natl. Acad. Sci. USA, 76:145 (1979)); and reverse-phase evaporation (REV) (Fraley et al., J. Biol. Chem., 255:10431 (1980); Szoka et al., Proc. Natl. Acad. Sci. USA 75:145 (1978); Schaefer-Ridder et al., Science, 215:166 (1982)), which are herein incorporated by reference.

Generally, the ratio of DNA to liposomes will be from about 10:1 to about 1:10. The ratio may also be from about 5:1 to about 1:5. More preferably, the ratio will be about 3:1 to about 1:3. The ratio may also be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,737, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055 and international publication No: WO 94/9469 (which are herein incorporated by reference) provide methods for delivering DNA-cationic lipid complexes to mammals.

Tumor cell antigens is defined as any cell surface antigen that is generally associated with tumor cells, i.e., occurring to a greater extent as compared with normal cells. Such antigens may be tumor specific. Alternatively, such antigens may be found on the cell surface of both tumorigenic and non-tumorigenic cells. These antigens need not be tumor specific. However, they are generally more frequently associated with tumor cells than they are associated with normal cells. Such tumor antigens that may be used as homing molecule (with respect to liposomes or in combination therapies) to direct the present invention to tumor cells include, but are not limited to, BAGE-1 (Boel, 1995), GAGE-1, 2, and 8 (Van den Eynde, 1995), GAGE 3-7 (DeBacker et al., 1999), GnTV (Guilloux, 1996), HERV K MeI (Schiavetti, 2002), KM-HN-1 (Monji, 2004), LAGE-1 (Rimoldi, 2000), MAGE-A1 (Traversari, 1992), MAGE-A2 (Chaux, 2001), MAGE-A3 (Gaugler, 1994), MAGE-A4 (Kobayashi, 2003), MAGE-A6 (Zorn, 1999), MAGE-A10 (Huang, 1999), MAGE-A12 (van der Bruggen, 1994b), MAGE-C2 (Ma, 2004), mucin (Jerome, 1993), NA-88 (Morea-Aubry, 2000), NY-ESO-1/LAGE-2 (Jager, 1998; Chen, 2000, Valmori, 2000), Sp17 (Chirivia-Internati, 2003), SSX-2 (Ayyoub, 2002), Trp2-Int2 (Lueptti, 1998), or peptide variants thereof in various positions of the above-described tumor specific antigens.

In one embodiment, nucleic acids comprising sequences encoding short hairpin RNA (shRNA) for seprase, are administered to treat, inhibit or prevent a disease or disorder associated with aberrant expression and/or activity of seprase via administration to a subject of an expressed or expressible nucleic acid. In this embodiment of the invention, the nucleic acids produce shRNA with complementarity to seprase, thereby preventing or reducing seprase gene expression to mediate a therapeutic effect.

Any of the methods for gene therapy available in the art may be used according to the present invention. Exemplary methods are described below.

For general reviews of the methods of gene therapy, see Goldspiel et al., Clinical Pharmacy 12:483-505 (1993); Wu and Wu, Biotherapy 3:87-95 (1991); Tolstoshev, Ann. Rev. Pharmacol Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which may be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the DNA encoding the shRNA seprase sequences are directly administered in vivo, where it is expressed to produce the shRNA. This may be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,236), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or nanospheres or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which may be used to target cell types specifically expressing the receptors). In another embodiment, nucleic acid-ligand complexes may be formed in which the ligand comprises a fusogenic viral peptide to disrupt endosomes, allowing the nucleic acid to avoid lysosomal degradation. In yet another embodiment, the nucleic acid may be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid may be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding shRNA of the invention are used. For example, a retroviral vector may be used (see Miller et al., Meth. Enzymol. 217:581-599 (1993)). These retroviral vectors contain the components necessary for the correct packaging of the viral genome and integration into the host cell DNA. The nucleic acid sequences encoding the shRNA to be used in gene therapy are cloned into one or more vectors, which facilitates delivery of the gene into a patient. More detail about retroviral vectors may be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. Genet. Devel. 3:110-114 (1993).

Adenoviruses are other viral vectors that may be used in gene therapy. Adenoviruses are especially attractive vehicles for delivering genes to respiratory epithelia. Adenoviruses naturally infect respiratory epithelia. Other targets for adenovirus-based delivery systems are liver, the central nervous system, endothelial cells, and muscle.

Adenoviruses have the advantage of being capable of infecting non-dividing cells. Kozarsky and Wilson, Curr. Opin. Genet. Develop. 3:499-503 (1993) present a review of adenovirus-based gene therapy. Bout et al., Human Gene Therapy 5:3-10 (1994) demonstrate the use of adenovirus vectors to transfer genes to the respiratory epithelia of rhesus monkeys.

Other instances of the use of adenoviruses in gene therapy may be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wana, et al., Gene Therapy 2:775-783 (1995).

In another embodiment, adenovirus vectors may be used. Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction may be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, and spheroplast fusion. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells may be delivered to a patient by various methods known in the art. The amount of cells envisioned for use depends such parameters as the desired effect, patient state, and may be determined by one skilled in the art.

Cells into which a nucleic acid may be introduced for purposes of gene therapy encompass any desired, available cell type, and include but are not limited to epithelial cells, endothelial cells, keratinocytes, fibroblasts, muscle cells, hepatocytes.

In an embodiment in which recombinant cells are used in gene therapy, nucleic acid sequences encoding shRNA preventing the expression of seprase are introduced into the cells such that they are expressible by the cells or their progeny, and the recombinant cells are then administered in vivo for therapeutic effect. In a specific embodiment, stem or progenitor cells are used. Any stem and/or progenitor cells that is capable of being isolated and maintained in vitro may be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

Another aspect of the present invention is directed to gene therapy methods for treating or preventing disorders, diseases and conditions. The gene therapy method of the present invention relate to the introduction of polynucleotide sequences into an animal to prevent the expression of seprase. This method requires a polynucleotide which encodes for an RNAi molecule operatively linked to a promoter and any other genetic elements necessary for the prevention of seprase expression within the target tissue.

Thus, for example, cells from a patient may be engineered with a polynucleotide comprising a promoter operably linked to a polynucleotide of the invention ex vivo, with the engineered cells then being provided to a patient to be treated with the RNAi.

In one embodiment, the polynucleotide of the invention is delivered as a naked polynucleotide. The term “naked” polynucleotide refers to sequences that are free from any delivery vehicle that acts to assist, promote or facilitate entry into the cell, including viral sequences, viral particles, liposome formulations, lipofectin, precipitating agents and/or similar agents of the like. However, the polynucleotide may also be delivered in liposome formulations and lipofectin formulations prepared by methods well known to those skilled in the art (e.g., U.S. Pat. Nos. 5,593,972, 5,589,466, and 5,580,859).

The polynucleotide vector constructs of the present invention used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art may be used for driving the expression of polynucleotide sequence of the invention. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; the U6 promoter (Paddison et al., Genes Development 16:948-958 (2002) and human growth hormone promoters.

Unlike other gene therapy techniques, one major advantage of introducing naked nucleic acid sequences into target cells is the transitory nature of the polynucleotide synthesis in the cells. Studies have shown that non-replicating DNA sequences may be introduced into cells to provide production of the desired polypeptide for periods of up to six months.

The lipid based delivery system harbours the polynucleotide construct of the invention that may be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of the polynucleotide will be in the range of from about 0.05 mg/kg body weight to about 50 mg/kg body weight. The dosage may be from about 0.005 mg/kg to about 20 mg/kg and preferably from about 0.05 mg/kg to about 5 mg/kg. Of course, as the artisan of ordinary skill will appreciate, this dosage will vary according to the tissue site of injection. The appropriate and effective dosage of nucleic acid sequence may readily be determined by those of skill in the art and may depend on the condition being treated and the route of administration.

A route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose.

The naked polynucleotides may be delivered by any method known in the art, including, but not limited to, direct needle injection at the delivery site, intravenous injection, topical administration, catheter infusion, and so-called gene guns. These delivery methods are known in the art.

The constructs may also be delivered with delivery, vehicles such as viral sequences, viral particles, liposome formulations, lipofectin, micropheres, nanopheres, precipitating agents, Such methods of delivery are known in the art.

In certain other embodiments, cells are engineered, ex vivo or in vivo, with polynucleotides of the invention contained in an adenovirus vector. Adenovirus may be manipulated such that it encodes and expresses the RNAi of the present invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz et al., Am. Rev. Respir. Dis., 109:233-238 (1974)). Finally, adenovirus mediated gene transfer has been demonstrated in a number of instances including transfer of alpha-1-antitrypsin and CFTR to the lungs of cotton rats (Rosenfeld et al., Science, 252:431-434 (1991); Rosenfeld et al., Cell, 68:143-155 (1992)). Furthermore, extensive studies to attempt to establish adenovirus as a causative agent in human cancer were uniformly negative (Green et al. Proc. Natl. Acad. Sci. USA, 76:6606 (1979)).

Suitable adenoviral vectors useful in the present invention are described, for example, in Kozarsky and Wilson, Curr. Opin. Genet. Devel., 3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224, which are herein incorporated by reference. For example, the adenovirus vector Ad2 is useful and may be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

The adenoviruses used in the present invention may be replication deficient. Replication deficient adenoviruses require the aid of a helper virus and/or packaging cell line to form infectious particles. The resulting virus is capable of infecting cells and may express a polynucleotide of interest which is operably linked to a promoter, but may not replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, Curr. Topics in Microbiol. Immunol., 158:97 (1992)). It is also one of the few viruses that may integrate its DNA into non-dividing cells. Vectors containing as little as 300 base pairs of AAV may be packaged and may integrate, but space for exogenous DNA is limited to about 4.5 kb. Methods for producing and using such AAVs are known in the art. See, for example, U.S. Pat. Nos. 5,139,941, 5,173,414, 5,354,673, 5,436,146, 5,474,935, 5,478,745, and 5,589,377.

For example, an appropriate AAV vector for use in the present invention will include all the sequences necessary for DNA replication, encapsidation, and host-cell integration. The polynucleotide construct containing polynucleotides of the invention is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, Appropriate helper viruses include adenoviruses, cytomegalovirus, vaccinia viruses, or herpes viruses. Once the packaging cells are transfected and infected, they will produce infectious AAV viral particles which contain the polynucleotide construct of the invention. These viral particles are then used to transduce eukaryotic cells, either ex vivo or in vivo. The transduced cells will contain the polynucleotide construct integrated into its genome, and will express the desired gene product.

Any mode of administration of any of the above-described polynucleotides constructs may be used so long as the mode results in the expression of shRNA against seprase in an amount sufficient to provide a therapeutic effect. This includes direct needle injection, systemic injection, catheter infusion, biolistic injectors, particle accelerators (i.e., gene guns), gelfoam sponge depots, other commercially available depot materials, osmotic pumps (e.g., minipumps), oral or suppositorial solid (tablet or pill) pharmaceutical formulations, and demayting or topical applications during surgery. For example, direct injection of naked calcium phosphate-precipitated plasmid into rat liver and rat spleen or a protein-coated plasmid into the portal vein has resulted in gene expression of the foreign gene in the rat livers. (Kaneda et al., Science, 243:375 (1989)).

A method of local administration is by direct injection. The polynucleotide of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient may undergo surgery and the polynucleotide construct may be coated on the surface of tissue inside the wound or the construct may be injected into areas of tissue inside the wound.

Methods of systemic administration, include intravenous injection, aerosol, oral and percutaneous (topical) delivery. Intravenous injections may be performed using methods standard in the art. Aerosol delivery may also be performed using methods standard in the art (see, for example, Stribling et al., Proc. Natl. Acad. Sci. USA, 189:11277-11281 (1992), which is incorporated herein by reference). Oral delivery may be performed by complexing a polynucleotide construct of the present invention to a carrier capable of withstanding degradation by digestive enzymes in the gut of an animal. Examples of such carriers, include plastic capsules or tablets, such as those known in the art. Topical delivery may be performed by mixing a polynucleotide construct of the present invention with a lipophilic reagent (e.g., DMSO) that is capable of passing into the skin.

Determining an effective amount of substance to be delivered may depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian. Therapeutic compositions of the present invention may be administered to any animal, preferably to mammals and birds. mammals include humans, dogs, cats, mice, rats, rabbits sheep, cattle, horses and pigs, with humans being particularly

The polynucleotides of the present invention are tested in vitro, and then in vivo for the desired therapeutic or prophylactic activity, prior to use in humans. For example, in vitro assays to demonstrate the therapeutic or prophylactic utility of the polynucleotides include, the effect of a compound on a cell line or a patient tissue sample. The effect of the polynucleotides on the cell line and/or tissue sample may be determined utilizing techniques known to those of skill in the art including, but not limited to, microarray analysis and cell lysis assays. In accordance with the invention, in vitro assays which may be used to determine whether administration of the polynucleotides is indicated, include in vitro cell culture assays in which a patient tissue sample is grown in culture, and exposed to or otherwise administered a compound, and the effect of such compound upon the tissue sample is observed.

The present invention also envisions the use of antibodies against seprase. The term antibody refers to an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Humanized antibodies of the invention include diabodies, tetrameric antibodies, single chain antibodies, tetravalent antibodies, multispecific antibodies (e.g., bispecific antibodies), domain-specific antibodies that recognize a particular epitope (e.g., antibodies that recognize an epitope bound by the antibody).

The term binding refers to an affinity between two molecules, for example, an antigen and an antibody. As used herein, specific binding means a preferential binding of an antibody to an antigen in a heterogeneous sample comprising multiple different antigens. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher. For example, specific binding of an antibody of the invention to a human seprase antigen includes binding in the range of at least about 1×10⁻⁷ to about 1×10⁻¹². Specific binding of an antibody of the invention to a human seprase also includes binding in the range of at least about 3×10⁻¹⁰ M to about 12×10⁻¹⁰ M, such as within the range of about 4×10⁻¹⁰ M to about 9×10⁻¹⁰ M, or such as within the range of about 7×10⁻¹⁰ M to about 12×10⁻¹⁰ M, or such as within the range of about 7×10⁻¹⁰ M to about 9×10⁻¹⁰ M, or such as within the range of about 9×10⁻¹⁰ M to about 12×10⁻¹⁰ M, or such as within the range of about 11×10⁻¹⁰ M to about 12×10⁻¹⁰ M, or greater binding affinities such as about 1.0×10⁻¹¹ M to about 10×10⁻¹¹ M, or about 1.0×10⁻¹¹ M to about 5×10⁻¹¹ M, or about 5.0×10⁻¹¹ M to about 10×10⁻¹¹ M. The phrase specifically binds also refers to selective targeting to seprase expressing cells when administered to a subject.

The term chimeric antibody is used herein to describe an antibody comprising sequences from at least two different species. Humanized antibodies are one type of chimeric antibody.

The term humanized is used herein to describe an antibody, wherein variable region residues responsible for antigen binding (i.e., residues of a complementarity determining region and any other residues that participate in antigen binding) are derived from a non-human species, while the remaining variable region residues (i.e., residues of the framework regions) and constant regions are derived, at least in part, from human antibody sequences. Residues of the variable regions and variable regions and constant regions of a humanized antibody may also be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species.

The present invention also provides for murine monoclonal anti-seprase antibodies and antibody fragments, and method for preparing and using the same. The anti-seprase antibodies mAb 65, mAb 68, mAb 82, and mAb 90 comprise at least one light chain or at least one heavy chain, or fragments thereof, wherein the anti-seprase antibody or antibody fragment (a) specifically binds to human seprase antigen with a binding affinity of at least about 1×10⁻⁷ M to about 1×10⁻¹² M; (b) specifically binds to human seprase antigen with a binding affinity greater than 1×10⁻¹¹ M; (c) specifically binds to human seprase antigen with a binding affinity greater than 5×10⁻¹¹ M; (d) specifically targets seprase-expressing cells in vivo; (e) competes for binding to human seprase with an antibody of any one of (a)-(d); (f) specifically binds to an epitope bound by any one of (a)-(d); or (g) comprises an antigen binding domain of any one of (a)-(d). The murine anti-seprase antibodies mAb 65, mAb 68, mAb 82, and mAB90 of the invention comprise constant regions that are derived from human constant regions, such as IgG1 or IgG4 constant regions.

Representative chimeric and humanized anti-seprase antibodies of the invention comprise at least one light chain or at least one heavy chain, or fragments thereof, wherein the chimeric or humanized anti-seprase antibody or antibody fragment (a) specifically binds to human seprase antigen with a binding affinity of at least about 1×10⁻⁷ M to about 1×10⁻¹² M; (b) specifically binds to human seprase antigen with a binding affinity greater than 1×10⁻¹¹ M; (c) specifically binds to human seprase antigen with a binding affinity greater than 5×10⁻¹¹ M; (d) specifically binds to human seprase antigen with a binding affinity greater than a binding affinity of murine mAB 65, mAb 68, mAb 82, and mAb 90 anti-seprase antibody binding to human seprase antigen; (e) specifically targets seprase-expressing cells in vivo; (f) competes for binding to human seprase antigen with an antibody of any one of (a)-(e); (g) specifically binds to an epitope bound by any one of (a)-(e); or (h) comprises an antigen binding domain of any one of (a)-(e).

Naturally occurring antibodies are tetrameric (H₂L₂) glycoproteins of about 150,000 daltons, composed of two identical light (L) chains and two identical heavy (H) chains. The two heavy chains are linked to each other by disulfide bonds and each heavy chain is linked to a light chain by a disulfide bond. Each of the light and heavy chains is further characterized by an amino-terminal variable region and a constant region. The term variable refers to the fact that certain portions of the variable domains differ extensively in sequence among antibodies and substantially determine the binding affinity and specificity of each particular antibody for its particular antigen. The variable regions of each of light and heavy chain align to form the antigen-binding domain.

Antibodies having a tetrameric structure, similar to naturally occurring antibodies, may be recombinantly prepared using standard techniques. Recombinantly produced antibodies also include single chain antibodies, wherein the variable regions of a single light chain and heavy chain pair include an antigen binding region, and fusion proteins, wherein a variable region of a humanized anti-seprase antibody is fused to an effector sequence, such as an Fc domain, a cytokine, an immunostimulant, a cytotoxin, or any other therapeutic protein. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561.

Tetravalent antibodies (H₄L₄) comprising two intact tetrameric antibodies, including homodimers and heterodimers, may be prepared for example as described in PCT International Publication No. WO 02/096948. Antibody dimers may also be prepared via introduction of cysteine residue(s) in the antibody constant region, which promote interchain disulfide bond formation, using heterobifunctional cross-linkers (Wolff et al. (1993) Cancer Res. 53: 2560-5), or by recombinant production to include a dual constant region (Stevenson et al. (1989) Anticancer Drug Des. 3: 219-30).

The term complementarity determining region or CDR refers to residues of the antibody variable regions that participate in antigen binding. A number of definitions of the CDRs are in common use. The Kabat definition is based on sequence variability, and the Chothia definition is based on the location of the structural loop regions. The AbM definition is a compromise between the Kabat and Chothia approaches.

The constant regions of the disclosed humanized anti-seprase antibodies are derived from constant regions from any one of IgA, IgD, IgE, IgG, IgM, and any isotypes thereof (e.g., IgG1, IgG2, IgG3, or IgG4 isotypes of IgG). The choice of the human isotype (IgG1, IgG2, IgG3, IgG4) and modification of particular amino acids in the human isotype may enhance or eliminate activation of host defense mechanisms and alter biodistribution of a humanized antibody of the invention. See (Reff et al. (2002) Cancer Control 9: 152-66).

Humanized antibodies may be prepared using any one of a variety of methods including veneering, grafting of complementarity determining regions (CDRs), grafting of abbreviated CDRs, grafting of specificity determining regions (SDRs). These general approaches may be combined with standard mutagenesis and synthesis techniques to produce an anti-seprase antibody of any desired sequence.

Grafting of CDRs is performed by replacing one or more CDRs of an acceptor antibody (e.g., a human antibody) with CDRs of a donor antibody (e.g., a non-human antibody). Acceptor antibodies may be selected based on similarity of framework residues between a candidate acceptor antibody and a donor antibody and may be further modified to introduce similar residues.

Analysis of the three-dimensional structures of antibody-antigen complexes, combined with analysis of the available amino acid sequence data was used to model sequence variability based on structural dissimilarity of amino acid residues that occur at each position within the CDR. See Padlan et al. (1995) FASEB J. 9: 133-139. Minimally immunogenic polypeptide sequences consisting of contact residues, which are referred to as specificity-determining residues (SDRs), are identified and grafted onto human framework regions.

Humanized anti-seprase antibodies of the invention may be constructed wherein the variable region of a first chain (i.e., the light chain variable region or the heavy chain variable region) is humanized, and wherein the variable region of the second chain is not humanized (i.e., a variable region of an antibody produced in a non-human species). These antibodies are referred to herein as semi-humanized antibodies. Anti-mAb 65, mAb-68, mb-82, and mb-90 murine antibodies may be used to prepare semi-humanized antibodies.

Variants of the disclosed murine and humanized anti-seprase antibodies may be readily prepared to include various changes, substitutions, insertions, and deletions, where such changes provide for advantages in use. For example, to increase the serum half life of the antibody, a salvage receptor binding epitope may be incorporated, if not present already, into the antibody heavy chain sequence. See U.S. Pat. No. 5,739,277. Other useful changes include substitutions as required to optimize efficiency in conjugating the antibody with a drug. For example, an antibody may be modified at its carboxyl terminus to include amino acids for drug attachment, for example one or more cysteine residues may be added. The constant regions may be modified to introduce sites for binding of carbohydrates or other moieties.

Variants of murine and humanized anti-seprase antibodies of the invention may be produced using standard recombinant techniques, including site-directed mutagenesis, or recombination methods.

In particular embodiments of the invention, anti-seprase variants are obtained using an affinity maturation protocol such as mutating the CDRs (Yang et al. (1995) J. Mol. Biol. 254: 392-403), chain shuffling (Marks et al. (1992) Biotechnology (NY) 10: 779-783), use of mutator strains of E. coli (Low et al. (1996) J. Mol. Biol. 260: 359-368), DNA shuffling (Patten et al. (1997) Curr. Opin. Biotechnol. 8: 724-733), phage display (Thompson et al. (1996) J. Mol. Biol. 256: 77-88), and sexual PCR (Crameri et al. (1998) Nature 391: 288-291).

For immunotherapy applications, relevant functional assays include specific binding to human seprase, internalization of the antibody when conjugated to a cytotoxin, and targeting to a tumor site(s) when administered to a tumor-bearing animal.

The present invention further provides cells and cell lines expressing humanized anti-seprase antibodies of the invention. Representative host cells include mammalian and human cells, such as CHO cells, HEK-293 cells, HeLa cells, CV-1 cells, and COS cells. Methods for generating a stable cell line following transformation of a heterologous construct into a host cell are known in the art. Representative non-mammalian host cells include insect cells (Potter et al. (1993) Int. Rev. Immunol. 10 (2-3):103-112). Antibodies may also be produced in transgenic animals (Houdebine (2002) Curr. Opin. Biotechnol. 13(6):625-629) and transgenic plants (Schillberg et al. (2003) Cell Mol. Life. Sci. 60(3):433-45).

Humanized anti-seprase antibodies of the invention also have utility in the detection of seprase in cells in vitro and in vivo based on their ability to specifically bind the seprase antigen. A method for detecting seprase-expressing cells may comprise: (a) preparing a biological sample comprising cells; (b) contacting a humanized anti-seprase antibody with the biological sample in vitro, wherein the antibody comprises a detectable label; and (c) detecting the detectable label, whereby seprase-expressing cells are detected.

The disclosed detection methods may also be performed in vivo, for example as useful for diagnosis, to provide intraoperative assistance, or for dose determination. Following administration of a labeled humanized anti-seprase antibody to a subject, and after a time sufficient for binding, the biodistribution of seprase-expressing cells bound by the antibody may be visualized. The disclosed diagnostic methods may be used in combination with treatment methods. In addition, humanized anti-seprase antibodies of the invention may be administered for the dual purpose of detection and therapy.

Representative non-invasive detection methods include scintigraphy (e.g., SPECT (Single Photon Emission Computed Tomography), PET (Positron Emission Tomography), gamma camera imaging, and rectilinear scanning), magnetic resonance imaging (e.g., convention magnetic resonance imaging, magnetization transfer imaging (MTI), proton magnetic resonance spectroscopy (MRS), diffusion-weighted imaging (DWI) and functional MR imaging (fMRI)), and ultrasound.

The present invention further relates to methods and compositions useful for inducing cytolysis of seprase-expressing cancer cells in a subject. Thus, the disclosed methods are useful for inhibiting cancer growth, including delayed tumor growth and inhibition of metastasis.

The present invention provides that an effective amount of a humanized anti-seprase antibody is administered to a subject. The term effective amount is used herein to describe an amount of a humanized anti-seprase antibody sufficient to elicit a desired biological response. For example, when administered to a cancer-bearing subject, an effective amount comprises an amount sufficient to elicit an anti-cancer activity, including cancer cell cytolysis, inhibition of cancer cell proliferation, induction of cancer cell apoptosis, reduction of cancer cell antigens, delayed tumor growth, and inhibition of metastasis. Tumor shrinkage is well accepted as a clinical surrogate marker for efficacy. Another well accepted marker for efficacy is progression-free survival.

For detection of seprase-expressing cells using the disclosed chimeric and humanized anti-seprase antibodies, a detectable amount of a composition of the invention is administered to a subject. A detectable amount, as used herein to refer to a diagnostic composition, refers to a dose of a chimeric or humanized H8 antibody such that the presence of the antibody may be determined in vitro or in vivo. For scintigraphic imaging using radioisotopes, typical doses of a radioisotope may include an activity of about 10 μCi to 50 mCi, or about 100 μCi to 25 mCi, or about 500 μCi to 20 mCi, or about 1 mCi to 10 mCi, or about 10 mCi. Actual dosage levels of active ingredients in a composition of the invention may be varied so as to administer an amount of the composition that is effective to achieve the desired diagnostic or therapeutic outcome. Administration regimens may also be varied. A single injection or multiple injections may be used. The selected dosage level and regimen will depend upon a variety of factors including the activity and stability (i.e., half life) of the therapeutic composition, formulation, the route of administration, combination with other drugs or treatments, the disease or disorder to be detected and/or treated, and the physical condition and prior medical history of the subject being treated.

For any anti-seprase or antibody/drug conjugate of the invention, the therapeutically effective dose may be estimated initially either in cell culture assays or in animal models, usually in rodents, rabbits, dogs, pigs, and/or or primates. The animal model may also be used to determine the appropriate concentration range and route of administration. Such information may then be used to determine useful doses and routes for administration in humans. Typically a minimal dose is administered, and the dose is escalated in the absence of dose-limiting cytotoxicity. Determination and adjustment of an effective amount or dose, as well as evaluation of when and how to make such adjustments, are known to those of ordinary skill in the art of medicine.

The invention provides methods of treatment, inhibition and prophylaxis by administration to a subject of an effective amount of the polynucleotides or pharmaceutical composition comprising the polynucleotides of the invention. In one aspect, the polynucleotides or composition is substantially purified (e.g., substantially free from substances that limit its effect or produce undesired side-effects). The subject is a mammal, including but not limited to animals such as cows, pigs, horses, chickens, cats, dogs, and primates, apes, chimps, rhesus monkey, and human.

Formulations and methods of administration that may be employed when the compound comprises a nucleic acid are described above; additional appropriate formulations and routes of administration may be selected from among those described herein below.

Various delivery systems are known and may be used to administer the polynucleotide or pharmaceutical compositions of the invention (i.e., nucleic acid encoding shRNAs specific for mammalian and human seprase gene), e.g., encapsulation in liposomes, microparticles, nanspheres microcapsules, recombinant cells capable of expressing the shRNA, receptor-mediated endocytosis (see, e.g., Wu and Wu, J. Biol. Chem. 262:4429-4432 (1987)), construction of a nucleic acid as part of a retroviral or other vector, Methods of introduction include but are not limited to intradermal, intramuscular, intraperitoneal, intravenous, subcutaneous, intranasal, epidural, and oral routes. The invention may be administered by any convenient route, for example by infusion or bolus injection, by absorption through epithelial or mucocutaneous linings (e.g., oral mucosa, rectal and intestinal mucosa) and may be administered together with other biologically active agents. Administration may be systemic or local. In addition, it may be desirable to introduce the polynucleotide or pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration may also be employed, e.g., by use of an inhaler or nebulizer, and formulation with an aerosolizing agent.

In a specific embodiment, it may be desirable to administer the polynucleotides or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the polynucleotide or pharmaceutical composition may be delivered in a vesicle, in particular a liposome (see Lanaer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid.)

In a specific embodiment where the shRNAs of the invention is a nucleic acid, the nucleic acid may be administered in vivo, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), Alternatively, the nucleic acid may be introduced intracellularly and incorporated within host cell DNA for expression, by homologous recombination.

The present invention also provides pharmaceutical compositions. Such compositions comprise a therapeutically effective amount of DNA encoding seprase shRNA and a pharmaceutically acceptable carrier. In a specific embodiment, pharmaceutically acceptable means approved by a regulatory agency of the Federal or a state government or listed in the U.S. Pharmacopeia or other generally recognized pharmacopeia for use in animals, and more particularly in humans. The term carrier refers to a diluent, adjuvant, excipient, or vehicle with which the therapeutic is administered. Such pharmaceutical carriers may be sterile liquids, such as water and oils, including those of petroleum, animal, vegetable or synthetic origin, such as peanut oil, soybean oil, mineral oil, sesame oil and the like. Water is a carrier when the pharmaceutical composition is administered intravenously. Saline solutions and aqueous dextrose and glycerol solutions may also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, may also contain minor amounts of wetting or emulsifying agents, or pH buffering agents. These compositions may take the form of solutions, suspensions, emulsion, tablets, pills, capsules, powders, sustained-release formulations and the like. The composition may be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, Examples of suitable pharmaceutical carriers are described in “Remington's Pharmaceutical Sciences” by E. W. Martin. Such compositions will contain a therapeutically effective amount of the RNAi together with a suitable amount of carrier so, as to provide the form for proper administration to the patient. The formulation should suit the mode of administration.

In a embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it may be dispensed with an infusion bottle containing sterile pharmaceutical grade water or saline. Where the composition is administered by injection, an ampoule of sterile water for injection or saline may be provided so that the ingredients may be mixed prior to administration.

The amount of the seprase shRNA which will be effective in the treatment, inhibition and prevention of a disease or disorder associated with aberrant expression and/or activity of seprase may be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) may be a notice in the form prescribed by a governmental agency regulating the manufacture, use or sale of pharmaceuticals or biological products, which notice reflects approval by the agency of manufacture, use or sale for human administration.

The present invention provides kits that may be used for the above methods. In one embodiment, a kit for treating a cancer patient by administering the vector of the present invention in order to suppress seprase expression.

Each of the polynucleotides identified herein may be used in numerous ways as reagents. The following description should be considered exemplary and utilizes known techniques.

The polynucleotide of the present invention may be used to control gene expression through triple helix formation or antisense DNA or RNA. Antisense techniques are discussed, for example, in Okano, J. Neurochem. 56: 560 (1991); “Oligodeoxynucleotides as Antisense Inhibitors of Gene Expression, CRCPress, Boca Raton, Fla. (1988). Triple helix formation is discussed in, for instance Lee et al., Nucleic Acids Research 6:3073 (1979): Cooney et al., Science 241: 456 (1988); and Dervan et al., Science 251:1360 (1991). Both methods rely on binding of the polynucleotide to a complementary DNA or RNA. For these techniques, polynucleotides are usually oligonucleotides 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense-Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein may be used to design antisense or triple helix polynucleotides in an effort to treat or prevent disease.

The present invention may also be used as a molecular biology tool. Additional seprase DNA sequences which may form shRNA loops may be inserted into the vector of the present invention to knockout seprase expression. Such sequences may completely abolish seprase expression at both the mRNA and protein level.

In another embodiment, the vector comprising the seprase DNA encoding for the shRNA may be used to identify genes involved in cancer progression, particularly, intravasation, extravasation and metastasis. Using, for example, microarray analyses, one may identify genes that are upregulated or downregulated by comparing the expression profiles of cells which express the seprase DNA encoding for the shRNA versus cells which do not. The cells which express the shRNA may be identified by the expression of a molecular marker, such as GFP.

In another embodiment, the vector of the present invention comprises a heterologous epitope tag or molecular marker which may be used to identify cells which harbour the vector. In the embodiment, the molecular marker is green fluorescent protein. Other markers and tags include, but are not limited to, other fluorescent proteins, epitope tags such as AU1, AU5, FLAG, myc, HA, VSV-G, and 6×HIS tags.

Polynucleotides of the present invention are also useful in gene therapy. One goal of gene therapy is to insert a new gene that was not present in the host genome, thereby producing a new trait in the host cell.

The polynucleotides of the present invention may be used in assays to test for one or more biological activities. Seprase exhibits enzymatic activity which results in the degradation of the extracellular matrix (ECM); it is therefore likely that seprase is be involved in the diseases associated with the biological activity, i.e., intravasation, metastasis, angiogenesis, Thus, the polynucleotides could be used to treat the associated disease.

Polynucleotides of the invention may be used to treat, prevent, and/or diagnose hyperproliferative diseases, disorders, and/or conditions, including cancer. Polynucleotides of the present invention may inhibit tumor cell invasion and proliferation through direct or indirect suppression of seprase expression, such as, but not limited to, by preventing seprase from heteromerizing with DPPIV or α3β1 integrin.

Examples of hyperproliferative diseases, disorders, and/or conditions that may be treated, prevented, and/or diagnosed by the polynucleotides of the present invention include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Other tumor cells, especially those which express seprase, may be targeted.

One embodiment utilizes the polynucleotide encoding the shRNA specific to the mammalian or human seprase gene of the present invention to inhibit aberrant cellular division and invasion (through the processes of angiogenesis, intravasation, and metastasis), by gene therapy using the present invention. The present invention provides a method for treating or preventing cell proliferative diseases, disorders, and/or conditions by inserting into an abnormally proliferating cell the DNA encoding the shRNA of the present invention, wherein said shRNA represses expression of seprase.

Polynucleotides of the present invention may be useful in inhibiting expression of oncogene genes or antigens. Inhibiting expression of the oncogenic genes, in the context of the present invention, is intended to mean the destruction of the seprase RNA, or the inhibition of the normal function of seprase. Normal functions of seprase include, but are not limited to, dimerizing with DPPIV/CD26, binding to α3β1 integrin, dual propyl dipeptidase activity, gelatinase activity, and degrading the ECM.

For local administration to abnormally proliferating cells, the DNA encoding the shRNAs of the present invention may be administered by any method known to those of skill in the art including, but not limited to transfection, electroporation, microinjection of cells, or in vehicles such as liposomes, micropheres, nanopheres, lipofectin, or as naked polynucleotides, or any other method described throughout the specification. The polynucleotide of the present invention may be delivered by known gene delivery systems such as, but not limited to, retroviral vectors (Gilboa, J. Virology 44:845 (1982); Hocke, Nature 320:275 (1986); Wilson, et al., Proc. Natl. Acad. Sci. U.S.A. 85:3014), vaccinia virus system (Chakrabarty et al., Mol. Cell. Biol. 5:3403 (1985) or other efficient DNA delivery systems (Yates et al., Nature 313:812 (1985)) known to those skilled in the art. These references are exemplary only and are hereby incorporated by reference. In order to specifically deliver or transfect cells which are abnormally proliferating and spare non-dividing cells, it is preferable to utilize a retrovirus, or adenoviral (as described in the art and elsewhere herein) delivery system known to those of skill in the art. Since host DNA replication is required for retroviral DNA to integrate and the retrovirus will be unable to self replicate due to the lack of the retrovirus genes needed for its life cycle. Utilizing such a retroviral delivery system for polynucleotides of the present invention will target said gene and constructs to abnormally proliferating cells and will spare the non-dividing normal cells.

The polynucleotides of the present invention may be delivered directly to cell proliferative disorder/disease sites in internal organs, body cavities and the like by use of imaging devices used to guide an injecting needle directly to the disease site. The polynucleotides of the present invention may also be administered to disease sites at the time of surgical intervention.

By cell proliferative disease is meant any human or animal disease or disorder, affecting any one or any combination of organs, cavities, or body parts, which is characterized by single or multiple local abnormal proliferations of cells, groups of cells, or tissues, whether benign or malignant.

Any amount of the polynucleotides of the present invention may be administered as long as it has a biologically inhibiting effect on the proliferation of the treated cells. Moreover, it is possible to administer more than one of the polynucleotides of the present invention simultaneously to the same site. By biologically inhibiting is meant partial or total growth inhibition as well as decreases in the rate of proliferation or growth of the cells. The biologically inhibitory dose may be determined by assessing the effects of the polynucleotides of the present invention on target malignant or abnormally proliferating cell growth in tissue culture, tumor growth in animals and cell cultures, or any other method known to one of ordinary skill in the art.

In another embodiment, the invention provides a method of delivering compositions to targeted cells. In another embodiment, the invention provides a method for the specific destruction of cells (e.g., the destruction of tumor cells) by administering the polynucleotides of the invention in association with toxins or cytotoxic prodrugs. By toxin is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By cytotoxic prodrug is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C. cytosine arabinoside, daunorubisin, and phenoxyacetamide derivatives of doxorubicin.

Therapeutic radiation may also be administered to the same tumor cell (or if in a patient, to the same cancer patient). Similarly anti-cancer chemotherapeutic agents may be administered to the same tumor cell or cancer patient. Such agents include: x-rays, cisplatin (Platinol®), daunorubicin (Cerubidine®), doxorubicin (Adriamycin®), etoposide (VePesid®)), methotrexate (Abitrexate®), mercaptopurine (Purinethol®), fluorouracil (Adrucil®), hydroxyurea (Hydrea®), Vinblastine (Velban®), Vincristine (Oncovin®), Irinotemay (Camptosar®, CPT-11), Levamisole, selective epidermal growth factor receptor tyrosine kinase inhibitors (e.g. ZD1839, Iressa.®) and Pacitaxel (Taxol®). Preferably the agents co-administered with the polynucleotides are ones that induce apoptosis.

As used herein, an anti-tumor drug means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), interferons and radioactive agents.

As used herein, a cytotoxin or cytotoxic agent means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, a chemotherapeutic regimen refers to any treatment with a chemotherapeutic agent. Examples of chemotherapeutic agents include, for example, the anti-tumor drugs listed above.

As used herein, a radioactive agent includes any radioisotope which is effective in destroying a tumor. Examples include, but are not limited to, cobalt-60 and X-ray emitters. Additionally, naturally occurring radioactive elements such as uranium, radium, and thorium which typically represent mixtures of radioisotopes, are suitable examples of a radioactive agent.

As used herein, administering means oral administration, administration as a suppository, topical contact, intravenous, intraperitoneal, intramuscular or subcutaneous administration, or the implantation of a slow-release device such as a miniosmotic pump, to the subject. As used herein, curing means to provide substantially complete tumor regression so that the tumor is not palpable.

The present invention provides for treatment of diseases, disorders, and/or conditions associated with neovascularization and intravasation by administration of the polynucleotides of the invention. Malignant and metastatic conditions which may be treated with the polynucleotides of the invention include, but are not limited to, malignancies, solid tumors, and cancers described herein and otherwise known in the art (for a review of such disorders, see Fishman et al., Medicine, 2d Ed., J. B. Lippincott Co., Philadelphia (1985)). Thus, the present invention provides a method of treating, preventing, and/or diagnosing an angiogenesis-related and intravasation-related diseases and/or disorders, comprising administering to an individual in need thereof a therapeutically effective amount of the polynucleotides of the invention. For example, DNA encoding the shRNAs of the present invention may be utilized in a variety of additional methods in order to therapeutically prevent intravasation and eventual metastasis of a cancer or tumor. Cancers which may be treated, prevented, and/or diagnosed with shRNAs against seprase, include, but are not limited to, solid tumors, including prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non-small cell lung cancer; colorectal cancer; and advanced malignancies. The DNA encoding the shRNAs may be delivered topically, in order to treat or prevent cancers such as skin cancer, head and neck tumors, breast tumors, and Kaposi's sarcoma.

Within yet other aspects, polynucleotides may be utilized to treat superficial forms of bladder cancer by, for example, intravesical administration. Polynucleotides may be delivered directly into the tumor, or near the tumor site, via injection or a catheter. Of course, as the artisan of ordinary skill will appreciate, the appropriate mode of administration will vary according to the cancer to be treated. Other modes of delivery are discussed herein.

Polynucleotides may be useful in treating, preventing, and/or diagnosing other diseases, disorders, and/or conditions, besides cancers, which involve angiogenesis and intravasation. These diseases, disorders, and/or conditions include, but are not limited to: benign tumors, for example hemangiomas, acoustic neuromas, neurofibromas, trachomas, and pyogenic granulomas; artheroscleric plaques; ocular angiogenic diseases, for example, diabetic retinopathy, retinopathy of prematurity, macular degeneration, corneal graft rejection, neovascular glaucoma, retrolental fibroplasia, rubeosis, retinoblastoma, uvietis and Pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within one aspect of the present invention methods are provided for treating, preventing, and/or diagnosing hypertrophic scars and keloids, comprising the step of administering the polynucleotide of the present invention to a hypertrophic scar or keloid.

Within one embodiment of the present invention, polynucleotides are directly injected into a hypertrophic scar or keloid, in order to prevent the progression of these lesions. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development. As noted above, the present invention also provides methods for treating, preventing, and/or diagnosing neovascular diseases of the eye, including for example, corneal neovascularization, neovascular glaucoma, proliferative diabetic retinopathy, retrolental fibroplasia and macular degeneration.

Ocular diseases, disorders, and/or conditions associated with neovascularization which may be treated, prevented, and/or diagnosed with the polynucleotides of the present invention include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal 22:291-312 (1978).

Since angiogenesis of other tissues is required and a prerequisite for intravasation, the present invention may be applied to other diseases such as ocular disease. Thus, within one aspect of the present invention methods are provided for treating or preventing neovascular diseases of the eye such as corneal neovascularization (including corneal graft neovascularization), comprising the step of administering to a patient a therapeutically effective amount of a the polynucleotide of the invention (as described above) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of diseases, disorders, and/or conditions may result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

Within particularly embodiments of the invention, may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic or anti-intrasvasation compositions, prepared as described above, may also be administered directly to the cornea. Within embodiments, the anti-angiogenic composition is prepared with a muco-adhesive polymer which binds to cornea. Within further embodiments, the anti-angiogenic factors or anti-angiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

Within other embodiments, the compounds described above may be injected directly into the corneal stroma by an ophthalmologist under microscopic guidance. The site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2-3 times per year. A steroid could also be added to the injection solution to reduce inflammation resulting from the injection itself.

Within another aspect of the present invention, methods are provided for treating or preventing neovascular glaucoma, comprising the step of administering to a patient a therapeutically effective amount of the polynucleotide to the eye, such that the formation of blood vessels is inhibited. In one embodiment, the compound may be administered topically to the eye in order to treat or prevent early forms of neovascular glaucoma. Within other embodiments, the compound may be implanted by injection into the region of the anterior chamber angle. Within other embodiments, the compound may also be placed in any location such that the compound is continuously released into the aqueous humor.

Within another aspect of the present invention, methods are provided for treating or preventing proliferative diabetic retinopathy, comprising the step of administering to a patient a therapeutically effective amount of the polynucleotide to the eyes, such that the formation of blood vessels is inhibited.

Within particularly embodiments of the invention, proliferative diabetic retinopathy may be treated by injection into the aqueous humor or the vitreous, in order to increase the local concentration of the polynucleotide in the retina. Preferably, this treatment should be initiated prior to the acquisition of severe disease requiring photocoagulation.

Within another aspect of the present invention, methods are provided for treating or preventing retrolental fibroplasia, comprising the step of administering to a patient a therapeutically effective amount of the polynucleotide to the eye, such that the formation of blood vessels is inhibited. The compound may be administered topically, via intravitreous injection and/or via intraocular implants.

In accordance with yet a further aspect of the present invention, there is provided a process for utilizing the polynucleotides of the invention for the purpose of wound healing. Polynucleotides may be clinically useful in preventing wound healing in situations involving surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from heat exposure or chemicals, and other abnormal wound healing conditions such as uremia, malnutrition, vitamin deficiencies and complications associated with systemic treatment with steroids, radiation therapy and anti-neoplastic drugs and antimetabolites. Polynucleotides of the invention, could be used to delay dermal reestablishment subsequent to dermal loss

The polynucleotides of the invention could be used to increase the adherence of skin grafts to a wound bed and to stimulate re-epithelialization from the wound bed. The following are a non-exhaustive list of grafts that polynucleotides of the invention could be used to increase adherence to a wound bed: autografts, artificial skin, allografts, autodermic graft, autoepdermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, thick split graft. The polynucleotides of the invention may be used to promote skin strength and to improve the appearance of aged skin.

It is believed that the polynucleotides of the invention will also produce changes in hepatocyte proliferation, and epithelial cell proliferation in the lung, breast, pancreas, stomach, small intestine, and large intestine. The polynucleotides of the invention could reduce or inhibit proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. The polynucleotides of the invention may reduce or inhibit the proliferation of endothelial cells, keratinocytes, and basal keratinocytes.

The polynucleotides of the invention could further be used in full regeneration of skin in full and partial thickness skin defects, including burns, (i.e., repopulation of hair follicles, sweat glands, and sebaceous glands), treatment of other skin defects such as psoriasis. The polynucleotides of the invention could be used to treat epidermolysis bullosa, a defect in adherence of the epidermis to the underlying dermis which results in frequent, open and painful blisters by accelerating reepithelialization of these lesions. The polynucleotides of the invention could also be used to treat gastric and duodenal ulcers and help heal by scar formation of the mucosal lining and regeneration of glandular mucosa and duodenal mucosal lining more rapidly. Inflammatory bowel diseases, such as Crohn's disease and ulcerative colitis, are diseases which result in destruction of the mucosal surface of the small or large intestine, respectively. Thus, the polynucleotides of the invention could be used to promote the resurfacing of the mucosal surface to aid more rapid healing and to prevent progression of inflammatory bowel disease. Treatment with the polynucleotides of the invention is expected to have a significant effect on the production of mucus throughout the gastrointestinal tract and could be used to protect the intestinal mucosa from injurious substances that are ingested or following surgery.

Due to seprase expression during embryonic development, the polynucleotides of the present invention may also increase or decrease the differentiation or proliferation of embryonic stem cells.

The above-recited applications have uses in a wide variety of hosts. Such hosts include, but are not limited to, human, murine, rabbit, goat, guinea pig, camel, horse, mouse, rat, hamster, pig, chicken, goat, cow, sheep, dog, cat, non-human primate, and human. In embodiments, the host is a mammal. In most embodiments, the host is a human.

All cited references are expressly incorporated herein in their entireties. While the invention has been described with respect to specific examples including presently modes of carrying out the invention, those skilled in the art will appreciate that there are numerous variations and permutations of the above described systems and techniques that fall within the scope of the invention as set forth in the appended claims.

### EXAMPLES

### Example 1

### Construction of pGUS Derived RNAi Vectors pGUS-SEP1384, pGUS-SEP1821, and pGUS-NO

A short hairpin RNA (shRNA) expression host vector, pGUS, was designed for the purpose of producing cells with green fluorescence protein (GFP) and suppressing seprase mRNA translation. Plasmid pGEM/U6 harboring a human U6 promoter sequence (Paddison et al., Genes Development 16:948-958 (2002) was kindly provided by Gregory J. Hannon (Cold Spring Harbor Laboratory, New York). The U6 promoter region, −˜500 to +1, was amplified by PCR with two primers: U6-upstream primer (5′-GGAACTCTAGTAACTAATTTAGGTGACACTATAGAATAC-3′) (SEQ ID NO: 1) and U6-downstream primer (5′GTCTTGACATGTCCGTAGGAAGACGCCGGTGTTTCGTCCTTTCCACAAGAT-3′) (SEQ ID NO: 2). A BpuAI site was integrated at the U6 transcriptional initiation site onto the U6-downstream primer to allow for the insertion of short hairpin RNA (shRNA) coding sequences. The PCR product was digested with MaeI (Roche) and BspLU 11I (Roche) and directionally cloned into plasmid pEGFP-C1 (Clontech) digested by BspLU 11I and AseI (BioLabs), whose recognition sites are located between the PUC plasmid replication origin and the human cytomegalovirus (CMV) immediate early promoter. Plasmid pEGFP-C1 expresses an enhanced GFP gene and a neomycin resistance gene. The resultant construct was propagated in E. coli TOP10F′ (Invitrogen) and named pGUS.

pGUS derived RNAi vectors for synthesis of shRNAs in mammalian cells were generated by inserting oligodeoxynucleotides encoding shRNAs and U6 terminator sequence into pGUS between the unique BpuAI and BspLU 11I sites. The sense strand sequence of shRNA was identified by first smayning the length of the target mRNA for an AAG sequence. These AAG related sequences (both the sense and antisense sequences) were also required to not have significant homology to other genes. Examples of the 20-nucleotide (20-nt) AAG related sequences identified from analysis of the human seprase gene are included in table one.¹ ¹Nucleotide positions of seprase cDNA human sequence (GenBank Accession No. U76833).

TABLE 1
Target Seprase shRNA Sequences

| Name of shRNA | AAG related sequence | | Region of Human |
|---|---|---|---|
| Sequence | from human seprase gene | SEQ ID NO: | Seprace cDNA |
| SEP-1384 | gaaaggtgccaatattacac | SEQ ID NO: 3 | 1384-1403 |
| SEP-1821 | gctgggtgtttatgaagttg | SEQ ID NO: 4 | 1821-1840 |
| SEP-1821v1 | cggtctgtatttgctgttaatt | SEQ ID NO: 5 | Variant of 1821-1840 |
| SEP-1821v2 | cgctgttaattggatatcttat | SEQ ID NO: 6 | Variant of 1821-1840 |
| SEP-1821v3 | cggctacaaacatattcactat | SEQ ID NO: 7 | Variant of 1821-1840 |
| SEP-1821v4 | cgcactcacactgaaggatatt | SEQ ID NO: 8 | Variant of 1821-1840 |
| SEP-2186 | agctctggttaatgcacaa | SEQ ID NO: 9 | 2186-2205 |
| SEP-2004 | tattacgcgtctgtctaca | SEQ ID NO: 10 | 2004-2023 |
| SEP-1421 | gtactatgcacttgtctgc | SEQ ID NO: 11 | 1421-1440 |
| SEP-1380 | aggtgccaatattacacag | SEQ ID NO: 12 | 1380-1399 |
| SEP-1380v1 | acatctacagaattagcat | SEQ ID NO: 13 | Variant of 1380-1399 |
| SEP-895 | tgatagcctcaagtgatta | SEQ ID NO: 14 | 895-914 |
| SEP-895v1 | tgatacggatataccagtt | SEQ ID NO: 15 | Variant of 895-914 |
| SEP-347 | ttacggcttatcacctgat | SEQ ID NO: 16 | 347-366 |
| SEP-346 | attacggcttatcacctga | SEQ ID NO: 17 | 346-365 |

The 20-nt sense strand sequence was inserted immediately downstream of the U6 promoter, followed by a spacer of 7-nt bearing a Hind III site, the 20-nt antisense strand sequence and the U6 terminator sequence (a string of 6 thymidines). Thus, the expression of encoded shRNA was controlled by U6 cassette and the G became the transcription initiating nucleotide of U6 transcripts.

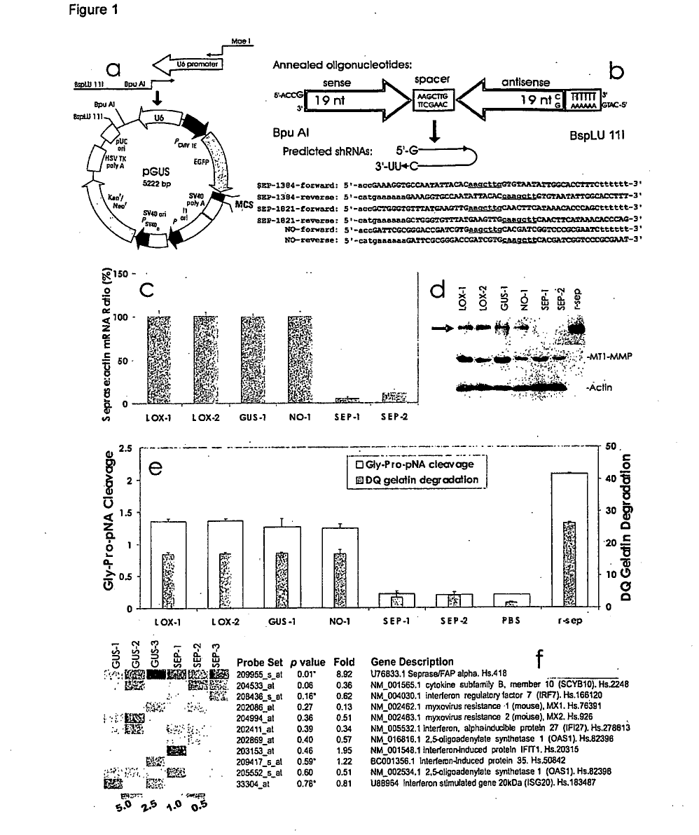

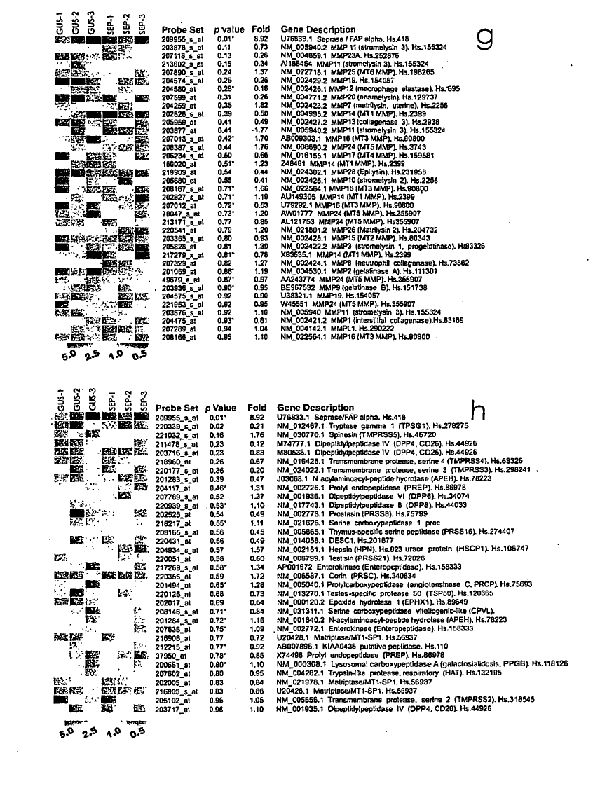

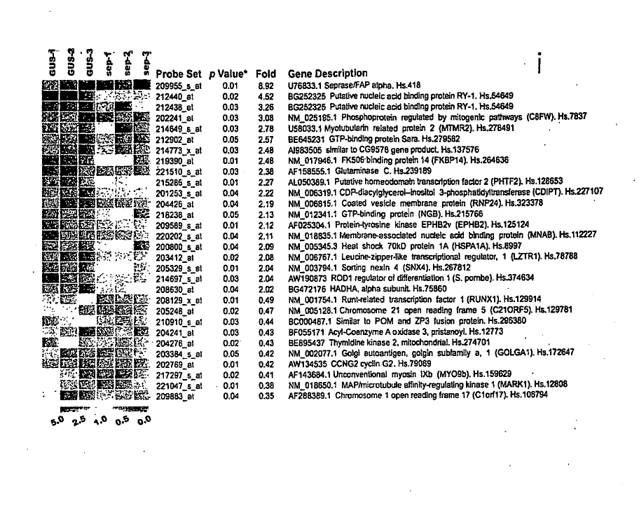

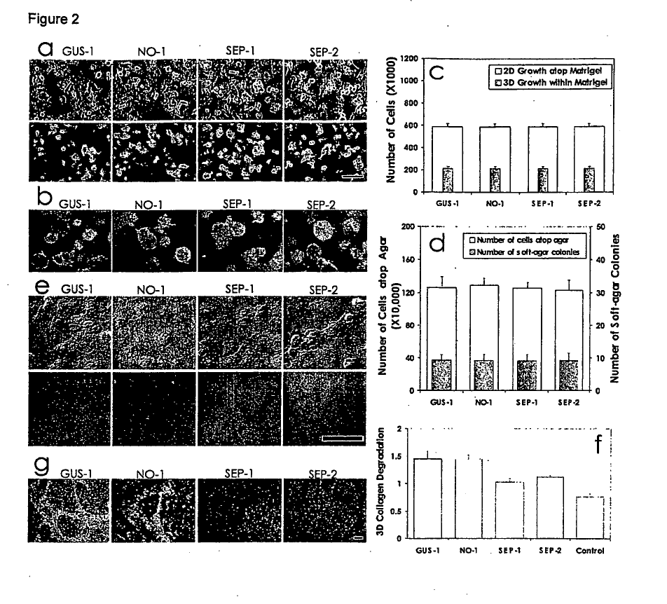

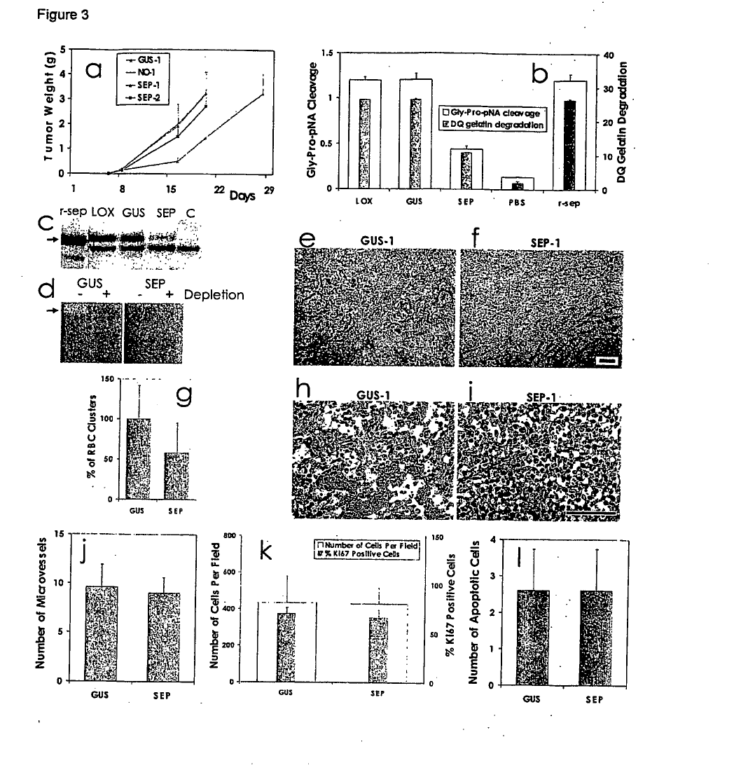

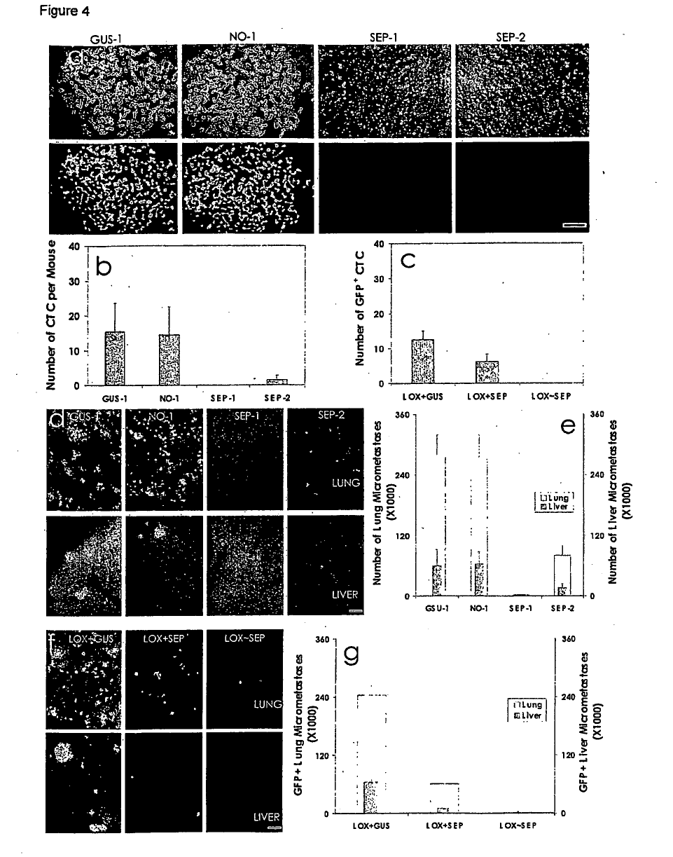

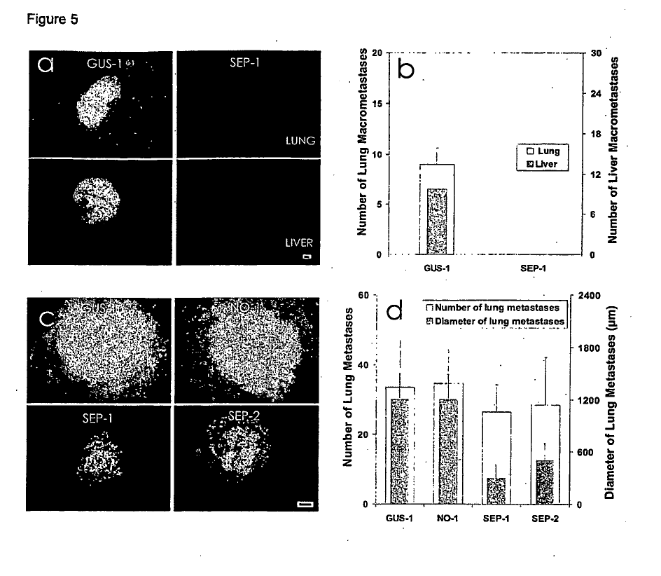

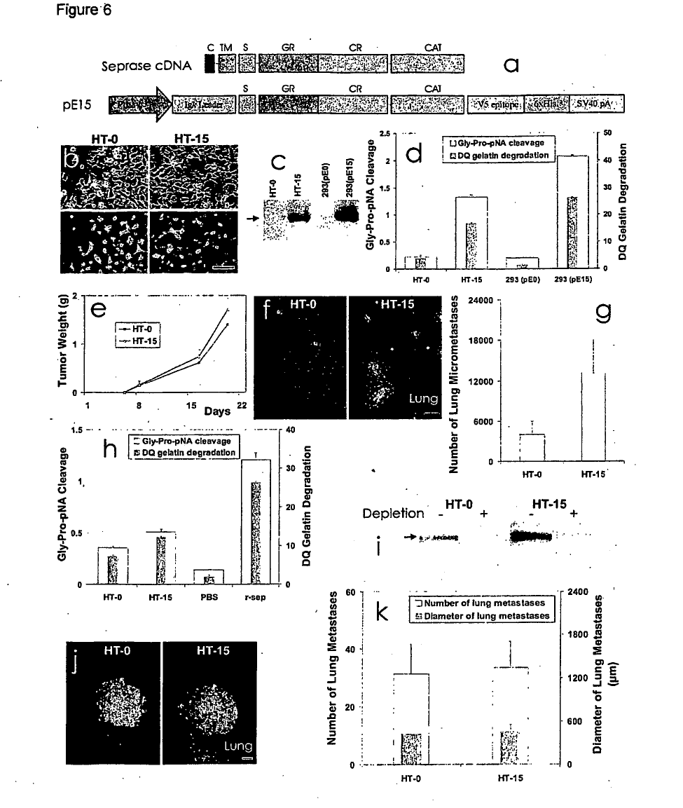

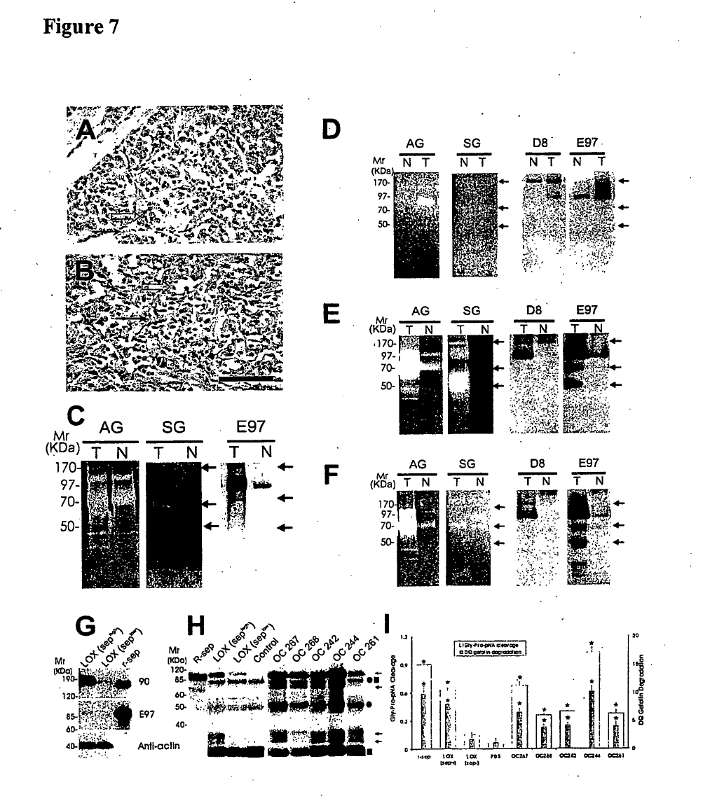

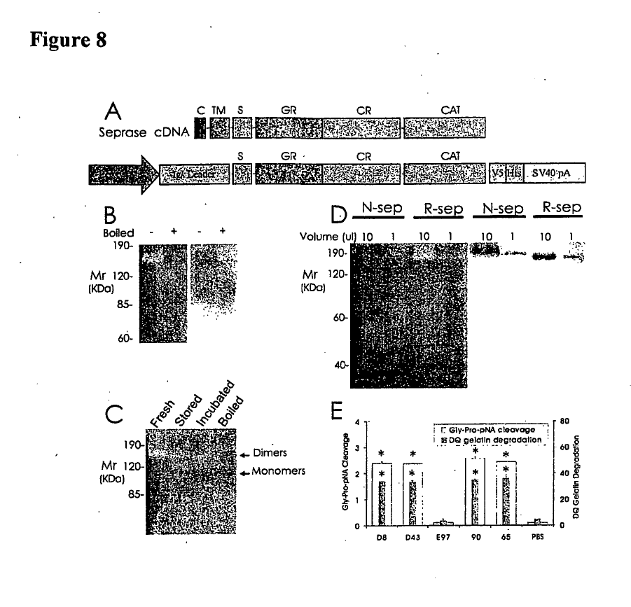

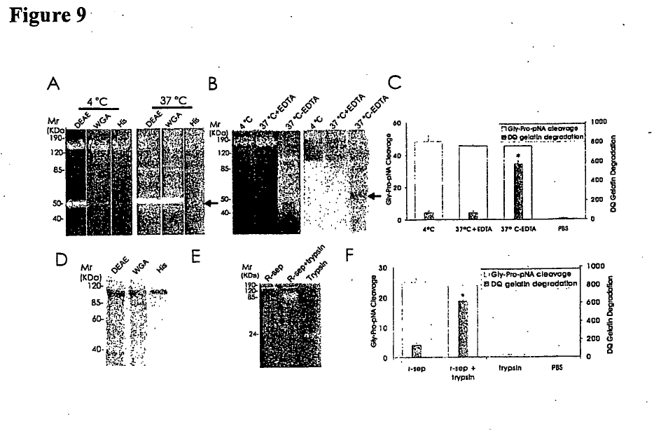

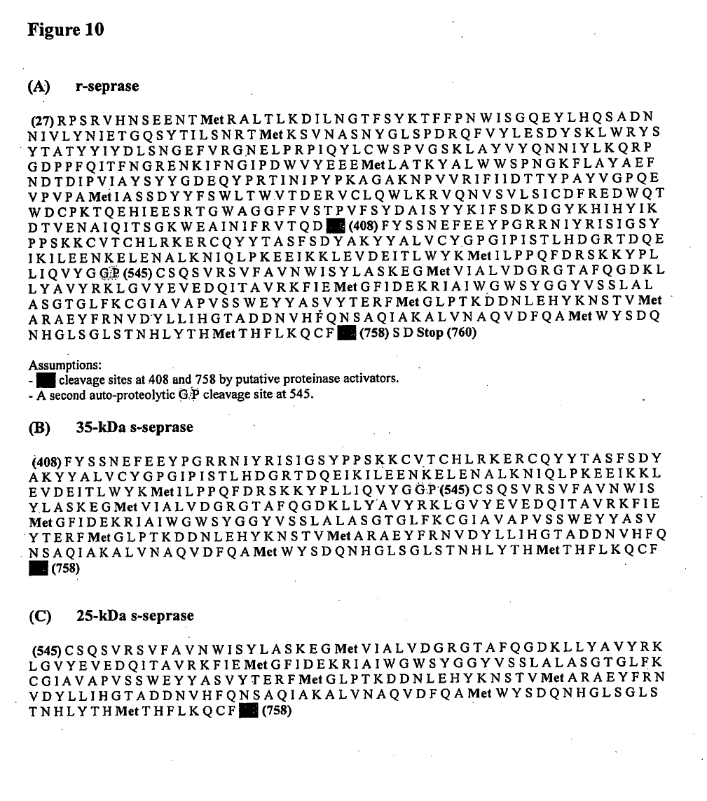

Two RNAi vectors were generated targeting different regions of seprase mRNA. These two vectors were designated pGUS-SEP1384 and pGUS-SEP1821 (Table 1). A control RNAi vector, designated pGUS-NO, was generated by cloning shRNA coding sequences immediately downstream of the U6 promoter (FIG. 1b). The control RNAi vector pGUS-NO targeted no human gene sequence.

### Example 2

### Selection of Suitable Cell Culture and Transfectants for Seprase Expression Studies

Particular cell lines of human amelanotic melanoma LOX cells (Fodstad et al., Int J Cancer 41:442-49 (1988)) were selected as host cells for studying seprase expression and activity based on a uniformly a high level of protein and proteolytic activities specific for seprase. In particular, the LOX-1 and LOX-2 cell lines were selected for their uniform expression of seprase. These cells were maintained in CCC medium [1:1 mixture of Dulbecco's modified Eagle's medium (GIBCO-BRL®) and RPMI 1640 medium (GIBCO-BRL®) supplemented with 10% calf serum (GIBCO-BRL®), 5% Nu-Serum IV Culture Supplement (BD Biosciences), and 2 mM L-glutamine]. Parental LOX sublines were single-cell cloned by limiting dilution and expanded in CCC medium conditioned by LOX cells. Cells of the LOX subline, LOX-1, were transfected with pGUS, pGUS-NO, pGUS-SEP1384, or pGUS-SEP1821 vectors using a LIPOFECTAMINE® Plus (GIBCO-BRL®) without linearization. Stable sublines were selected by CCC medium supplemented with G418 (600 μg/ml) for sublines with green fluorescence, followed by one more cycle of single-cell cloning and designated GUS, NO, SEP-1, or SEP-2 subline, respectively. SEP-1 harbors pGUS-SEP1384 and SEP-2 harbors pGUS-SEP1821. Among these stable sublines, approximately half were fluorescently green and selected for further characterization. The expression of GFP was independent of seprase expression (data not shown).

Real-time PCR experiments were conducted to determine the whether the short hairpin double stranded RNA seprase loops produced by pGUS-SEP1384 or pGUS-SEP1821 would interfere with the post-transcriptional endogenous seprase mRNAs in the parent LOX-1 cells. Total RNA for real-time RT-PCR was purified from 1×10⁶ cells by RNeasy Mini Kit (Qiagen) and RNA (1.0 μg) was reverse-transcribed by 1st Strand cDNA Synthesis Kit for RT-PCR (AMV, Roche) with random primers supplied by the kit. To measure seprase mRNA levels, two primers were designed, (5′-TTGCCATCTAAGGAAAGAAAGG-3′) (SEQ ID NO: 18) and (5′-TTTTCTGACAGCTGTAATCTGG-3′) (SEQ ID NO: 19), against a unique region of human seprase cDNA, as confirmed by BLAST, and whose expected PCR product was 499-bp. To evaluate RNAi efficiency, these two primers were also designed to generate a PCR product spanning the shRNA targeting sites (Martinez et al., Cell 110:563-74 (2002)). To quantify β-actin mRNA levels, two primers: (5′-AGATGACCCAGATCATGTTTGA-3′) (SEQ ID NO: 20) and (5′-GCACAGCTTCTCCTTAATGTCA-3′) (SEQ ID NO: 21), were used to generate an endogenous control with a PCR product of 300-bp. PCR reactions were set up with LightCycler-FastStart DNA Master SYBR Green I Kit (Roche). The cDNA was diluted 5-fold and 2 μl was added to each PCR reaction as template. Real-time PCR was performed by LightCycler System (Roche) equipped with LightCycler Software Version 3 (Roche). To generate standard curves, the cDNA derived from the parental LOX-1 subline was diluted in 5-fold increments and used in parallel PCR reactions. Melting curves were generated to confirm the specificity of PCR products. Quantification was performed in duplicate.

Real-time PCR revealed that 32 of 62 SEP sublines generated by pGUS-SEP1384 and 16 of 28 SEP sublines generated by pGUS-SEP1821 had seprase mRNA knocked down or reduced but 32 GUS sublines and 32 NO sublines did not. A typical real-time PCR result is shown in FIG. 1c. In contrast with parental cells LOX-1 and LOX-2 and control GUS and NO cells, SEP-1 cells reduced more than 95% of seprase mRNA and SEP-2 cells more than 80%.

Western immunoblotting analysis and proteolytic immunocapture assays were performed to detect seprase protein and seprase prolyl dipeptidase and gelatinase activities as described in Ghersi et al., 2002. Western immunoblotting analysis showed no reactivity in seprase mRNA suppressed cells (FIG. 1d, SEP-1 and SEP-2), whereas positive reaction was found in LOX-1 (no pGUS related vector), LOX-2 (no pGUS related vector), GUS and NO cells and control proteins, including membrane type 1-matrix metalloproteinase (MT1-MMP) and actin, remain unaltered (FIG. 1d, lower two panels). To examine seprase expression in LOX sublines, cells were harvested with cooled 10 mM EDTA in PBS, pH 7.4, and cell lysate was prepared with cooled RLN buffer [140 mM NaCl, 1.5 mM MgCl₂, 0.5% (v/v) NP-40 in 50 mM Tris-HCL, pH 8.0]. Cells expressing high seprase protein, including LOX-1, LOX-2, GUS-1 and NO-1 cells, exhibited high levels of seprase-specific prolyl dipeptidase and serine gelatinase activities, whereas cells expressing low seprase protein, including SEP-1 and SEP-2 cells, had low levels of seprase-specific proteolytic activities (FIG. 1e). These cells with specific alteration of seprase expression remained stable for the period of experiments (3 months).

### Example 3

### DNA Microarray Analysis Defining Specificity of Seprase RNA Suppression

DNA microarray analysis was used to further define the specificity of RNAi seprase suppression in cell sublines generated. Proteins such as matrix metallaproteinase-2 (MMP-2) and membrane type 1 metallaproteinases (MT1-MMP/MMP-14) were proposed as important mediators for growth and metastasis of malignant melanomas (Seftor et al., Cancer Res 61:6322-27 (2001)). Also, several MMPs and serine proteases have been shown to be involved in melanoma invasion (Bittner et al., Nature 406:536-40 (2000); Seftor et al., Cancer Res 61:6322-27 (2001); Monsky et al., Cancer Res 53:3159-64 (1993); Nakahara et al., Proc Natl Acad Sci USA 94:7959-64 (1997); Artym et al., Carcinogenesis 23:1593-1601 (2002)). Total RNA from LOX sublines was purified by RNeasy Mini Kit (Qiagen). Generation of cRNA, labeling, hybridization and smayning of the Affymetrix high-density oligonucleotide microarray chip (Hu133A, 22,283 probe sets), were performed according to the manufacturer's instruction (Affymetrix). Absolute analysis of each chip was carried out using the Affymetrix Microarray Suite 5.1 Software to generate raw expression data. Statistical analysis was performed using GeneSpring 6.0 software (Silicon Genetics), in which raw data were normalized to 50th percentile per chip then to the median per gene. Also, GeneSpring 6.0 was used to investigate the variation in gene expression, show clusters of coordinately expressed genes, and indicate the relationships between cell sublines. For genes of interest, expression fold change was calculated with mean expression levels within the control cell group (GUS-1 to 3 sublines) and the seprase suppressed cell group (SEP-1 to 3 sublines). The expression level of each gene in the samples was indicated by a shade-coded scheme, in which the various shades of black and grey are proportional to the fold change from the median of the GUS and SEP cell samples. Based on log-transformed data and with an active cross-gene error model, significance of expression change for each gene was evaluated using Welch's t test without assumption of equality of variances (Kang et al., Cancer Cell 3:537-49 (2003)).

Absolute expression patterns of interferon target genes (FIG. 1f), matrix metalloproteinase (MMP) genes (FIG. 1g), serine protease genes (FIG. 1h) and potential regulatory genes associated were compared with altered seprase expression (FIG. 1i) in GUS sublines and SEP sublines. The seprase gene expression was specifically knocked down and the translation was prevented in SEP sublines compared with GUS sublines. In contrast, the expression of 9 interferon target genes, 22 MMP genes, and 30 serine protease genes were unaffected in all six sublines examined (FIGS. 1g and 1h). These serine protease genes, including 3 probe sets for DPP4/CD26 (a seprase homologue) and a recently reported tumor associated serine protease, matriptase/MT1-SP1, were not altered in the seprase-suppressed SEP sublines as compared with the seprase-expressing GUS sublines. The gene expression analysis of the cell sublines shows that the stable sublines generated with pGUS-SEP RNAi vectors have, so far, produced very low non-specific gene suppression. Also, seprase function in tumor intravasation is specific in the background of major proteases commonly found in melanoma.

In order to identify a novel set of genes responsive to seprase RNAi knockdown, DNA microarray analysis was performed to compare gene expression patterns of GUS sublines, which expressed seprase, with that of SEP sublines, which exhibited seprase suppression. The comparisons only highlighted 18 genes up-regulated in the GUS sublines and 10 genes up-regulated in the SEP sublines (FIG. 1g and 1i). This analysis shows that melanoma cells with suppressed seprase expression down-regulate 18 other genes, including putative nucleic acid binding protein RY-1, phosphoprotein regulated by mitogenic pathways (C8FW), myotubularin related protein 2 (MTMR2), and GTP-binding protein Sara; these cells also up-regulate 10 other genes, including chromosome 1 open reading frame 17 (C1orf17), MAP/microtubule affinity-regulating kinase 1 (MARK1), unconventional myosin 1Xb (MYO9b), CCNG2 cyclin G2, Golgi antoantigen golgin subfamily a 1 (GOLGA1) and mitochondrial thymidine kinase 2 (FIG. 1i). These genes were not detected in previous DNA microarray studies on whole melanoma tissue (Bittner et al., Nature 406:536-40 (2000)).

The length of the small interfering RNA (siRNAs) designed for reducing seprase translation also did not affect other genes like the 9 interferon target genes. Although shRNAs predicted from the pGUS-SEP vectors were thought to be too short to induce interferon response, a substantial number of shRNA vectors and siRNAs could trigger an interferon response (Bridge et al., Nat Genet 34:263-64 (2003); Sledz et al., Nat Cell Biol 5:834-39 (2003), Heidel et al., Nat Biotechnol 22:1579-82 (2004); Kim et al., Nat Biotechnol 22:321-25 (2004); Hornung et al., Nat Med 11:263-70 (2005)). As discussed above, the DNA microanalysis in FIG. 1f shows that seprase gene expression was specifically knocked down or the translation was prevented in SEP sublines compared with GUS sublines whereas the expression of 9 interferon target genes were unaffected in all six sublines examined.

### Example 4

### Physiological and Behavioral Differences of Cells with Altered Seprase Expression in Culture

The physiological and behavioral differences of cells were investigated between LOX cells with high seprase expression (GUS-1, NO-1) and those LOX cell lines with inhibited seprase expression via prevention of mRNA translation (SEP-1 and SEP-2). When cultured on 2D plain plastic surface (FIG. 2a) or atop 2D Matrigel, cells with contrasting levels of seprase expression have identical cell morphology and indistinguishable cell proliferation rates (FIGS. 2a; 2c). When cultured in 3D Matrigel, these sublines also showed similar cell shape and proliferation rates (FIGS. 2b; 2c). Likewise, these cells grow equally well on and in type I collagen gel (data not shown) and soft agar (FIG. 2d).

However, the SEP-1 and SEP-2 cell lines which had altered seprase expression, behaved differently in their interaction with the extracellular matrix. In vitro cell invasion assays were performed to determine cell invasiveness by using FITC fibronectin coated, crosslinked gelatin films as described previously (Chen et al., J Tiss Cult Meth 16:177-81 (1994)). GFP labeled cells were post-fixed with 20% formaldehyde (that bleached the GFP fluorescent signal) and photographed by differential interference contrast microscopy. The films were photographed using epifluorescence microscopy.

When cultured on fibronectin-coated gelatin films (Chen et al., J Tiss Cult Meth 16:177-81 (1994)), seprase expressing GUS-1 and NO-1 sublines invaded locally at invadopodia sites on fibronectin-coated gelatin films. In contrast, seprase-suppressed SEP-1 and SEP-2 cells could not (FIG. 2e). In parallel, RNAi suppression of α3β1 integrin expression in LOX cells did not alter this cellular ability (data not shown). When seeded on top of 3D gels of Matrigel or type I collagen at high cell density and incubated for 10 days (Hendrix et al., Nat Rev Cancer 3:411-21 (2003); Maniotis et al., Am J Pathol 155:739-52 (1999)), seprase-expressing GUS and NO cells formed vasculogenic-like networks, but seprase-suppressed SEP cells (SEP-1 and SEP-2) could not (FIG. 2g).

Further evidence that seprase contributes to cellular invasiveness was obtained using cell based collagen degradation assays (Ghersi et al., 2002). When embedded in 3D gels of TRITC-labeled type I collagen (Id at 2002), seprase-expressing GUS-1 and NO-1 cells released significantly more TRITC-peptides from the gels than SEP-1 and SEP-2 cells (FIG. 2f). These datas show that seprase-expressing cells are highly invasive than those cells with low expression of seprase.

### Example 5

### Behavior of Cells with Altered Seprase Expression in Primary Tumors

The physiological consequences of seprase were then studied using spontaneous and experimental metastasis models. Specifically, seprase's contribution to tumor intravasation was investigated and in turn, the effect of tumor intravasation on promoting growth of the primary tumor with increasing vasculogenic mimicry and subsequent metastasis of tumor cells.

For the spontaneous metastasis model, LOX and HT1080 sublines (6×10⁵ cells per injection) were injected subcutaneously (s.c.) into both flanks of 4-8 Fox Chase SCID mice (Taconic; female) per cell subline (GUS-1, NO-1, SEP-1, and SEP-2). To introduce seprase-expressing cells into seprase-suppressed tumors, SCID mice were bilaterally co-inoculated s.c. with LOX-1 cells (3×10⁵ cells) and SEP-1 cells (3×10⁵ cells) per side; control mice were either bilaterally co-inoculated with LOX-1 cells (3×10⁵ cells) and GUS-1 cells (3×10⁵ cells) per side or injected with 6×10⁵ LOX-1 cells on one side and 6×10⁵ SEP-1 cells on the other side. Tumors were measured once a week. After 20 days, mice were sacrificed by anesthetic overdose and tumors were excised and weighed. The ratio of total tumor weight to tumor volume was calculated. Tumor weight during earlier days was estimated by the following equation: tumor weight (g)=tumor length (cm)×tumor width (cm)×tumor height (cm)×0.3251 (g/cm³).

Using a spontaneous metastasis model, 6×10⁵ cells per injection were inoculated s.c. to both flanks of eight SCID mice per cell subline. All cells could produce local tumors within eight days that showed similar latent period and size (FIG. 3a). However, tumor size was noticeable different among those derived from cells with high and low seprase expression when tumors reached 18 days after inoculation (FIG. 3a). Accordingly, large tumors derived from GUS sublines exhibited higher levels of proteolytic activities (FIGS. 3b; 3d) and protein (FIG. 3c) specific for seprase than small tumors from SEP sublines.

Histological staining of tissue sections was used to study effect of seprase expression on vascularization of tumor cells. In the histological analysis, tissues were fixed in 10% neutral buffered formalin and routinely processed and then embedded in paraffin. Tissue sections were prepared and, after deparaffinization with three washes in xylene, the sections were rehydrated through a series of graded ethanols (100%, 90% and 70%) to water before staining with hematoxylin and eosin. Histological staining of tissue sections revealed that large GUS tumors (FIGS. 3e; 3g; 3h) contained more vasculatures lined with tumor cells and filled with red blood cells, indicating vasculogenic mimicry (Hendrix et al., 2003), than small SEP tumors (FIGS. 3f; 3g; 3i). However, immunostaining of tumor sections with a panel of endothelial cell markers, including von Willebrand Factor, CD31 and CD34 showed that the density of microvessels in GUS and SEP tumors were similar (FIG. 3j). In addition, tumor and stromal cells appear similarly in both seprase-expressing GUS and seprase-suppressed SEP tumors, including relative cell density and percentage of proliferating cells (FIG. 3k), and number of apoptotic cells (FIG. 31).

These results on the primary tumors derived from cells with contrasting levels of seprase expression (GUS-1, NO-1, SEP-1, and SEP-2) support a mechanism for seprase function in tumor intravasation. The proteolytic activities specific for seprase expressed on melanoma and fibrosarcoma cells locally degrade their surrounding extracellular matrices to allow blood vessel and tumor growth. These data show that seprase plays a central role for vasculogenic mimicry in melonoma cells (Hendrix et al., Nat Rev Cancer 3:411-21 (2003)). In addition, seprase inhibits angiogenesis in breast carcinoma cells (Huang et al., Cancer Res 64:2712-2716 (2004)).

In addition, these results show that tumors of approximately 3 grams derived from cells with high seprase contained more vasculatures lined with tumor cells and filled with red blood cells than those derived cells with low seprase activity. Furthermore, tumors derived from mixed LOX-1 and SEP-1 cells contained enhanced vasculogenic mimicry than SEP-1 tumors (data not shown). Cumulatively, these data demonstrate that cells with high seprase invade toward blood more rapidly than cells with low seprase, and large GUS tumors are readily accessible to blood supplies that, in turn, enhance proliferation of seprase-expressing cells.

### Example 6

### Tumor Cells with High Seprase Expression May Intravasate

It has been shown that tumor cells quickly die in a few hours after entering the circulation, a process known as metastatic inefficiency (Hoffman, Nat Rev Cancer 5:796-806 (2005)). It was found, however, that a majority of tumor cells survived in the capillary network in secondary organs for long periods of time to form metastatic colonies (Al Mehdi et al., 2000). Accordingly, to determine whether tumor cells have entered the circulation, a fibronectin-coated type 1 collagen film (Ghersi et al., J Biol Chem 277:29231-41 (2002)) was used to enrich circulating tumor cells followed by culturing cells in a medium conditioned by parental cells and containing G418 for 14 days to develop GFP-LOX colonies.

Specifically, for the experimental metastasis model, LOX and HT1080 sublines were injected intravenously (i.v.) through the tail vein (3.0×10⁵ cells/mouse) in 3 female FOX Chase SCID mice per cell line. After 22 days, mice were sacrificed by anesthetic overdose. To study the circulating tumor cells in each SCID mouse that was injected s.c. with LOX or HT1080 cell sublines, at the time the mouse was sacrificed, blood (0.7 ml/mouse) was collected through heart in the presence of anti-coagulants and mononuclear cells were fractionated using Ficoll-Paque Plus (Amersham Pharmacia Biotech) density gradient centrifugation. In order to differentially expand the circulating tumor cells, mononuclear cells were seeded on a 10-cm plate layered with fibronectin-coated type I collagen films (Ghersi et al., J Biol Chem 277:29231-41 (2002)) and cultured in the medium conditioned by parental cells and containing G418. After 14 days, colonies derived from circulating tumor cells were identified by epifluorescence microscopy and counted.

GUS and NO tumors with high seprase activity developed tumor colonies in blood. SEP-1 tumors with 95% of seprase mRNA suppressed developed no tumor colonies in the blood. SEP-2 tumors with 80% of seprase mRNA suppressed have less than 10% tumor colonies developed from blood as compared to the GUS and NO injected mice (FIGS. 4a; 4b). When non-fluorescence-labeled, seprase-expressing LOX-1 cells were mixed with GFP-labeled, seprase-suppressed GUS-1 or SEP-1 cells (3×10⁵ cells per subline) and co-inoculated s.c. into both flanks of SCID mice, the LOX-1+GUS-1 tumor generated twice as many GUS-1 colonies in blood as GUS-1 tumor (6×10⁵ cells per injection) and the LOX-1+SEP-1 tumor produced as many SEP-1 colonies in blood as GUS-1 tumor (FIGS. 4b; 4c). In addition, when non-fluorescence-labeled, seprase-expressing LOX-1 cells and GFP-labeled, seprase-suppressed SEP-1 cells were inoculated s.c. separately into each flank (6×10⁵ cells per injection) of SCID mice, no GFP positive SEP-1 colony was found in the blood (FIG. 4c). These data show that tumors derived from cells with high seprase expression intravasate and produce circulating tumor cells but tumors from cells with less than 5% of seprase expression do not. Also, introduction of cells with high seprase with these with low seprase into same tumor site could drive the tumor cells with low seprase to intravasate.

To determine the number of circulating tumor cells that were capable of surviving over a few weeks during the metastatic process, the relative number of solitary GFP-tagged micrometastases (tumor clusters containing less than five cells) were measured in the lung and liver. To measure and visualize these GFP tagged metastases, tissues including lung, liver, spleen and heart were removed and directly examined by epifluorescence microscopy, followed by enumeration and photography. Consistent with the above finding on circulating tumor cells, mice bearing the tumors derived from seprase-expressing GUS and NO cells generated more lung and liver micrometastases than mice carrying tumors derived from seprase-suppressed SEP cells (FIGS. 4d; 4e). When non-fluorescence-labeled, seprase-expressing LOX-1 cells were mixed with GFP-labeled, seprase-expressing GUS-1 or GFP-labeled, seprase-suppressed SEP-1 cells (3×10⁵ cells per subline) and co-inoculated s.c. into both flanks of SCID mice, the LOX-1+GUS-1 tumor generated twice as many lung and liver micrometastases as GUS-1 tumor (6×10⁵ cells per injection) and the LOX-1+SEP-1 tumor produced as many lung and liver micrometastases as GUS-1 tumor (FIGS. 4d; 4e; 4f; 4g). When non-fluorescence-labeled, seprase-expressing LOX-1 cells and GFP-labeled, seprase-suppressed SEP-1 cells were inoculated s.c. separately into each flank (6×10⁵ cells per injection) of SCID mice, there is GFP expressing, SEP-1 micrometastases found in the lung and liver (FIGS. 4f; 4g). These data show that the presence of cells with high seprase expression is essential for formation of lung and liver micrometastases in this model.

### Example 7

### Tumor Cells with Altered Expression Form Metastatic Growth

To examine if the presence of micrometastases in the circulation observed above may develop into metastatic outgrowth in the lung and liver, primary tumors from a set of four mice for each cell subline were surgically removed at day 20 after s.c. inoculation and macrometastases presented in the lung and liver (clumps containing more than 10 tumor cells) were examined after an additional 20 days. Mice bearing GUS-1 tumors develop macrometastases in the lung and liver, whereas mice carrying SEP-1 tumors do not (FIGS. 5a; 5b). To clarify whether seprase is involved in metastatic outgrowth in secondary organ sites, an experimental metastasis model was used wherein approximately equal number of cells with altered seprase expression was directly introduced into the circulation via tail vein injection. We found that, 10 minutes after the i.v. injection, the majority of melanoma cells were arrested in lung, and 20 days later, cells with high seprase (GUS-1 and NO-1) or low seprase (SEP-1 and SEP-2) produced similar number of macrometastases in the lung (FIGS. 5c; 5d). However, size of metastatic colonies correlated with levels of seprase expression of injected cells: GUS-1 and NO-1 cells with high seprase produced large metastatic colonies, SEP-2 cells with 80% of seprase mRNA suppressed formed colonies of intermediate size, and SEP-1 cells with greater than 95% of seprase mRNA suppressed generated the smallest macrometastases. These data implicate the involvement of seprase in metastatic outgrowth, probably through promoting tumor interaction with blood for nutrient supplies.

Examples 4-7 demonstrate that seprase is required for tumor invasion and contact with blood to promote active growth of primary and secondary tumors. Seprase was a characteristic cell invasive phenotype by conferring tumor cells with the ability of locally invading fibronectin-coated gelatin films, degrading type 1 collagen gel, and forming vasculogenic networks in culture (see FIG. 2). In the spontaneous and experimental metastasis models of Examples 6 and 7, it was shown that seprase contributes to tumor intravasation, which, in turn, promotes growth of primary tumors by increasing vasculogenic mimicry and metastasis with escalating number of circulating and solitary tumor cells in the circulation and enlargement of metastatic colonies (FIGS. 3-5). Neither cell proliferation in 2D and 3D cultures in vitro nor tumor growth latency period in vivo were affected by the altered expression of seprase in tumor cells. Seprase however, confers the enhanced growth of primary and secondary tumors by its invasion function that promotes blood supply to tumor cells.

### Example 8

### Role of Seprase in Non-Melanoma Types

To determine whether seprase plays a role of tumor intravasation in cancer types other than melanoma, fibrosarcoma cells were generated with high seprase expression using an overexpression vector from human fibrosarcoma HT 1080 line that contains no or low detectable seprase protein.

Vector pE0 was originally modified from plasmid pCEP4 (Invitrogen) that contained a hygromycin resistance gene for selection of stable transfectants. To over-express active seprase, vector pE15 was constructed by inserting the coding sequence of seprase entire extracellular domain (amino acids 27-760) downstream of the CMV promoter between an N-terminal mouse Igk secretion signal and a C-terminal V5-His tag into the pE0 vector. Seprase cDNA encoding the predicted cytoplasmic domain and transmembrane domain were excluded in the pE15 vector.

To establish HT1080 sublines that stably express GFP and seprase, cells were transfected with pGUS, followed by selection with CCC medium supplemented with G418 (600 μg/ml). Stable sublines with green fluorescence were mixed and further transfected with pE0 or pE15. Sublines that stably express GFP and seprase were selected by CCC medium supplemented with Hygromycin B (200 μg/ml, GBCO-BRL) and were pooled. 293-EBNA (Invitrogen), an engineered host for pE15 that over-expresses seprase, was used as a control line to express seprase.

The behavior of the fibrosarcoma cells with contrasting levels of seprase expression in cell culture and the metastasis model were further examined. Plasmid pE15 was constructed to express the extracellular domain of seprase (FIG. 6a), whereas a control empty vector was designated pE0. HT1080 cells were sequentially transfected with vector pGUS and pE15 to generate stable HT-15 subline that expressed both GFP and seprase. Similarly, control HT-0 subline was obtained by double transfection of pGUS and pE0 vector (FIG. 6b). To detect seprase expressed by double transfected HT1080 cells, cell culture media was used. Compared with HT-0 cells, HT-15 cells expressed higher levels of protein and proteolytic activities specific for seprase (FIGS. 6c and 6d). When inoculated s.c. into SCID mice, HT-15 cells produced slightly bigger tumors than HT-0 cells (FIG. 6e). Importantly, there were significantly more lung micrometastases in the mice bearing HT-15 tumors than in mice carrying HT-0 tumors (FIGS. 6f and 6g). Consistently, higher levels of protein and proteolytic activities specific for seprase were detected in primary tumors derived from HT-15 cells than from HT-0 cells (FIGS. 6h and 6i). When HT-0 and HT-15 cells were inoculated i.v. into SCID mice, both HT-15 and HT-0 cells could form equal number of metastatic colonies, but the colonies derived from HT-15 cells were slightly bigger than that from HT-0 cells (FIGS. 6j and 6k). Taken together, these data demonstrate that seprase promotes intravasation and post-angiogenic growth of both primary tumors and metastases when it is over-expressed in fibrosarcoma cells.

### Example 9

### Identification and Characterization of Truncated Seprase

Native human seprase (n-seprase) is a 170-kDa transmembrane glycoprotein with gelatinolytic activity. The identification of a 70 to 50-kDa truncated human seprase (s-seprase) was investigated. Immunohistochemical technique was performed to evaluate the expression of seprase in surgically removed tumor proper and adjacent tissues (melanoma, invasive breast carcinoma, colon and stomach carcinoma and ovarian carcinoma) using a panel of mAbs: D8, D28 and D43, that recognized seprase as described (Iwasa et al., Cancer Lett. 227:229-236 (2005); Jin et al., Anticancer Research 23:3195-3198 (2003); Mori et al., Oncology 67:411-419 (2004); Okada et al., Oncology 65:363-370 (2003)). FIGS. 7A and 7B show an example of malignant melanoma positive immuno-staining against cellular nuclei background counterstained by hematoxylin. Control samples only show hematoxylin stains. Seprase was detected in each of the stained tissues (i.e., melanoma, invasive breast carcinoma, colon and stomach carcinoma and ovarian carcinoma tissues).

Wheat germ agglutin binding proteins (WGA-binding proteins) were used to isolate seprase from tumor tissues in order to determine whether seprase had gelatin degradation activity. The WGA-binding proteins were purified from paired tumor and adjacent tissues from patients and analyzed in parallel by gelatin zymography and immunoblotting using mAbs D8 (directed against seprase dimer and monomer) and E97 (against seprase monomer and polypeptide fragments) as described in Mori et al., Oncology 67:411-419 (2004) and Okada et al., Oncology 65:363-370 (2003).

Results of the parallel gelatin zymograph and immunoblotting of seprases isolated from WGA-binding proteins found seprases of three sizes (170 kDa, 70 kDa, and 50 kDa) in melanoma (FIG. 7C) and carcinomas of the breast (FIG. 7D), colon (FIG. 7E), and stomach (FIG. 7F), respectively. This direct comparison of gelatinolytic activities and proteins specific for seprase shows that: (1) in the conditions to resolve all types of gelatinases (AG in FIGS. 7C-7F), major gelatinolytic activities co-purified with seprase by WGA column from tissue lysates are matrix metalloproteinases (MMPs) and they are more prominent in tumors than in adjacent normal tissues; (2) in the conditions that MMPs were suppressed by EDTA to resolve serine type gelatinase activities (SG in FIGS. 7C-7F), gelatinolytic activities and proteins specific for seprase are found to be more prominent in tumors than in adjacent normal tissues; (3) gelatinolytic activities of 70- to 50-kDa proteins that were recognized by mAb E97 directed against seprase subunits and polypeptides are the s-seprase specific for tumors (FIGS. 7C-7F). These data demonstrate the existence of s-seprase with increasing gelatinase activity in malignant melanoma, invasive ductal carcinoma of the breast, and adenocarcinoma of the colon and stomach.

Seprase was also immuno-affinity-purified from xerographs derived from various LOX melanoma cell lines (GUS-1, NO, SEP-1, SEP-2) with contrasting levels of seprase expression. The subunit composition of s-seprase was determined in human tumors developed in immuno-deficient nude mice (FIGS. 7G-7I). LOX cells with low seprase expression were generated by RNA interference (RNAi) knockdown using the vector pGUS-SEP1384 (SEP-1, followed by stable cell selection, and cells with high seprase was initiated used control pGUS vector (GUS-1, NO) (See Examples 1 and 2) (FIG. 7G). Tumors derived from cells with high seprase expression produced protease subunits of 35- to 25-kDa but these from cells with low seprase did not (FIG. 7H). The former also exhibited higher dipeptidyl peptidase (DP) and gelatinase activities than the latter (FIG. 7I).

Seprase was also affinity-purified from 5 cases of malignant adenocarcinomas of the ovary (FIGS. 7H-7I). All ovarian tumors contained protease subunits of 35- to 25-kDa (FIG. 7H) and exhibited higher DP and gelatinase activities than the control tumor with low seprase expression (FIG. 7I). These data show that n-seprase has a 170-kDa gelatinase activity with 97-kDa subunits, whereas s-seprase with 70- to 50-kDa gelatinase activities occurs in all tumors examined and composes of 35- to 25-kDa subunits. The amino acid sequences for the 35 kDa s-seprase (SEQ ID NO: 22) is shown in FIG. 10b. The amino acid sequence for the 25 kDa s-seprase (SEQ ID NO: 23) is shown in FIG. 10c.

### Example 10

### Expression of a Recombinant Seprase Lacking the Transmembrane Domain

A novel recombinant seprase was designed for purposes of studying enhanced geletinase activity and as a possible therapeutic (wound healing, burns etc). The recombinant seprase (r-seprase) was designed from the native human seprase gene wherein the nucleotides encoding the transmembrane domain was deleted and a nucleotide sequence encoding a secretion signal was added. The pA15 plasmid, which contains the human seprase cDNA sequence (GenBank Accession No. U76833) was utilized as a PCR template. The cDNA fragment encoding the seprase extracellular domain (amino acids 27-760) was amplified with a forward primer (5′ AAGGATCCCGCCCTTCAAGAGTTCATAACT 3′) (SEQ ID NO: 24) and a reverse primer (5′ AACTCGAGGTCTGACAAAGAGAAACACTG 3′) (SEQ ID NO: 25). The PCR product, excluding the coding sequences of seprase short cytoplasmic domain (amino acids 1-6) and hydrophobic transmembrane domain (amino acids 7-26), was inserted into a modified pCEP4 vector (Invitrogen), an Epstein-Barr Virus (EBV) based vector employing the cytomegalovirus (CMV) immediate early enhancer/promoter for high level transcription of recombinant genes and carrying an EBV replication origin (oriP) to permit its extrachromosomal replication in human cells. In comparison with pCEP4, the modified vector additionally harbored the encoding sequences of a secretion signal from the V-J2-C region of the mouse Ig kappa-chain and a V5-His fusion tag, derived from pSecTag/FRT/V5-His-TOPO vector (Invitrogen). The cDNA sequence of seprase extracellular domain was inserted in frame with the N-terminal secretion signal and the C-terminal V5-His tag, allowing efficient secretion, easy detection (by Anti-V5 Antibody; Invitrogen) and rapid purification (by His•Bind Resin columns; Novagen) of recombinant seprase. The final construct was verified by sequencing both DNA strands and was named pE15.

Plasmid pE15 was transfected into 293-EBNA cells using LIPOFECTAMINE® Reagent (GBCO-BRL®) according to the manufacturer's instruction.293-EBNA monkey kidney cells (Invitrogen), which are intended for use with vectors containing an EBV origin of replication (oriP), were maintained in DMEM (GBCO-BRL®) supplemented with 10% fetal bovine serum (GBCO-BRL®) and 250 μg/ml G418 under 5% CO₂. After transfection, cells were cultured initially in DMEM containing 10% fetal bovine serum, 250 μg/ml G418 and 200 μg/ml Hygromycin B (GBCO-BRL®), then cultured in protein-free HyQ PF-293 medium (HyClone) containing 250 μg/ml G418 and 200 μg/ml Hygromycin B. Cell viability was checked with Trypan Blue (GBCO-BRL®) exclusion test. Freshly collected culture medium was filtered with four layers of filter paper (Whatman) at room temperature and loaded onto an equilibrated DEAE Sepharose Fast Flow column (SIGMA®) at 4° C. R-seprase was eluted with a NaCl gradient from 0 M to 1.0 M in 10 mM phosphate buffer, pH 7.0, and then absorbed by a WGA affinity chromatography column (Amersham Pharmacia Biotech). After being eluted with 0.5 M N-Acetylglucosamine (SIGMA®) in PBS, r-seprase was further purified by the charged His•Bind Resin column (Novagen). The following elution was performed either with the elution buffer containing 1 M imidazole or with the stripping buffer containing 0.1 M EDTA. Eluted protein was concentrated to 400 μl with an ULTRAFREE-15 Centrifugal Filter Device (Millipore) and fractionated with a Superdex 200 Prep grade gel filtration column (Pharmacia Biotech). In the entire procedure, r-seprase was tracked by the soluble DP assay.

Like the 170-kDa n-seprase (Goldstein et al., Biochemica 1361:11-19 (1997); Pineiro-Sanchez et al., J. Biol. Chem. 272:7595-7601 (1997)), the r-seprase has an 160-kDa dimer with gelatinase activity that may be dissociated into two 90-kDa subunits under storage and temperatures greater than 60° C. (FIGS. 8B-8C). Parallel SDS PAGE analyses on purified r-seprase using gelatin zymography and Western immunoblotting showed that the 160-kDa dimer degraded gelatin but the 90-kDa monomer could not degrade gelatin (FIG. 8C). The amino acid sequence of the 80-kDa r-seprase protein (SEQ ID NO: 26) is shown in FIG. 10A. The full length sequences of the 90 kDa n-seprase is available under GenBank Accession No. AAC51668 (SEQ ID NO: 27).

### Example 11

### Design of Novel Antibodies to R-Seprase and Characterization of the Extacellular Domain of Seprase

Antibodies specific to the novel r-seprase protein were designed as follows. BABL/c inbred mice (Taconic) were immunized using 10 μg of purified r-seprase. Splenocytes and Sp2/0-Ag14 cells (ATCC) were fused. Hybridomas were screened with ELISA. Briefly, Microtiter® U bottom Polyvinyl Chloride 96 well plates (Dynex Technologies) were coated with r-seprase and blocked with 5% BSA in PBS. Coated r-seprase was subjected to a reaction with Hybridoma supernatant and Anti-Mouse IgG Peroxidase-Conjugates (Sigma) sequentially. The bound secondary antibody was detected with 2,2′-Azino-bis(3-ethylbenzthiazoline-6-sulfonic acid) Diammonium Salt solution (Sigma) and recorded by absorbance at 410 nm with a Microplate Spectrophotometer System equipped with SOFTmax Pro version 1.3.1 (Molecular Devices). Hybridomas secreting mAbs against r-seprase were further confirmed by soluble enzymatic assays and Western immunoblotting analyses. MAb isotype was determined by ImmunoType Mouse Monoclonal Antibody Isotyping Kit (Sigma). MAb 65, mAb 68, mAb 82 and mAb 90 were created as described above. The monoclonal antibodies mAb 65, mAb 68, mAb 82, and mAb 90 are all immunoglobulin isotype IgG1. These monoclonal antibodies were prepared by culturing hybridoma cells in Cellgro Protein Free Medium (Mediatech) and isolated with a Protein G Sepharose 4 column (Amersham Pharmacia Biotech) and a Superdex 200 Prep Grade Gel Filtration column.

The 160-kDa dimer isolated by mAbs D8, D43, 90 or 65 exhibited significantly higher DP and gelatinase activities than the dissociated subunits and fragments isolated by mAb E97 (FIG. 8E). In contrast to the previous proposed role of the transmembrane domain in holding subunits together (Goldstein et al., Biochemica 1361:11-19 (1997); Pineiro-Sanchez et al., J. Biol. Chem. 272:7595-7601 (1997)), the present data demonstrate that the cytoplasmic and transmembrane domains of seprase are not required for its subunit dimerization and for its enzymatic activities.

A comparison n-seprase and r-seprase as shown in FIG. 2D demonstrates that the gelatinase activity of membrane bound n-seprase is strongly inhibited. However, previous investigations utilized detergents to extract the membrane bound n-seprase. Detergents have the ability to alter the 3-D structure of proteins and, possibly in the case of n-seprase, open the protein to increase the availability of a catalytic site, which, in vivo, would be inaccessible. In this way, the methods of extraction of the membrane bound n-seprase may confer the detected catalytic activity, whereas the membrane bound n-seprase in its cellular environment may not exhibit any activity until it is proteolytically activated.

### Example 12

### Proteolytic Truncation of Seprase Activates its Gelatinase Activity but not DP Activity

N-terminal truncation of seprase activates its gelatinase activity but does not affect its DP activity. Firstly, the gelatinase activity of n-seprase and r-seprase (FIG. 8D). Both membrane-bound n-seprase and the N-terminal truncated r-seprase were immunoaffinity-purified using the mAb 90, which recognizes the dimers of both n-seprase and r-seprase (FIG. 8D). Although more proteins were found to associate with n-seprase from the detergent soluble cell lysate as described in Ghersi et al., J. Biol. Chem., 277:29231-29241 (2002) than r-seprase from the medium conditioned by cells, the gelatinase activity of the r-seprase dimer is considerably higher than that of the n-seprase dimer (FIG. 8D). This greater activity is a result of truncation of the native form.

Proteolytic truncation of r-seprase and its associated increase in gelatinase activity occur at 37° C. (FIG. 9A). The gelatinase activation occurs readily in r-seprase purified by DEAE Sepharose and WGA affinity chromatography columns that contain greater than 10% of impurity (FIG. 9D), but not in r-seprase purified by His•Bind Resin column that has over 99% purity (FIG. 9D). The gelatinase activity was seen as a ladder of gelatinolytic bands, ranging from 100- to 50-kDa with the 50-kDa prominent band on gelatin zymograms (FIG. 9A). These smaller gelatinases decrease in fractions with increasing purification: DEAE>WGA>His, suggesting the involvement of other components for seprase activation.

Activation of the 160-kDa r-seprase into the 50-kDa gelatinase involve an EDTA-sensitive endogenous enzymatic activator (FIG. 9B). When the r-seprase samples partially purified by DEAE Sepharose and WGA-affinity chromatography columns were incubated at 4° C. or 37° C. for 1 day in the presence or absence of 5 mM EDTA (FIG. 9B, indicated by 37° C.+EDTA and 37° C.−EDTA, respectively), and then subjected to gelatin zymography and Western immunoblotting using mAb E97, the 50-kDa gelatinase activity and protein specific for seprase only appeared in the r-seprase sample incubated at 37° C. and without EDTA treatment (FIG. 9B). Similarly, when the r-seprase samples prepared above were subjected to the soluble enzymatic assays for DP and gelatinase activities specific for seprase, the r-seprase sample incubated at 37° C. and without EDTA treatment exhibits a seven-fold increase in gelatinase activity without altering DP activity (FIG. 9C). Incubation of r-seprase at 4° C. and in the presence of EDTA does not significantly change the DP and gelatinase activities of r-seprase (FIG. 9C).

The truncation of r-seprase by trypsin treatment may increase its gelatinase activity but not its DP activity (FIGS. 9E; 9F). Trypsinized r-seprase shows higher gelatinase activity than pure r-seprase and trypsin on a gelatin zymogram (FIG. 9E). Trypsinized r-seprase also has a five-fold increase in gelatinase activity than pure r-seprase as determined by a soluble enzymatic assay; however, there was no significant difference in the DP activity detected. These data demonstrate that proteolytic truncation of seprase reduces steric hindrance for the gelatin substrate, but not the DP substrate, and increases the gelatinolytic activity of seprase.

Overall, the gelatinase activity of the 50-kDa seprase was elevated as shown by gelatin zymography (FIGS. 9A; 9B) and soluble DP and gelatinase assays (FIG. 9C). However, the DP activity of different forms of r-seprase was not increased by proteolytic truncation (FIGS. 9C; 9F), indicating that the DP catalytic domain of all forms of seprase remains active. Interestingly, anti-V5 antibody and His•Bind Resin column were not able to capture the truncated 50-kDa form (data not shown). Since the anti-V5 antibody and His•Bind Resin column are able to bind to the full-length r-seprase dimer and monomer, it is possible that the 50-kDa, truncated r-seprase might have lost its C-terminal V5 and His tags during the proteolytic cleavage and production of the shorter form.

### Example 13

### Method of Treating Burn Victims

It will be appreciated that conditions caused by a burns or wounds wherein tissue requires time for regeneration can be treated by administering a therapeutic dosage of the either r-seprase or s-seprase of the present invention. Thus, the invention also provides a method of treatment of an individual in need of an increased level of the polypeptide comprising administering to such an individual a pharmaceutical composition comprising an amount of the polypeptide to increase the activity level of the polypeptide in such an individual to provide time for regeneration of tissue and prevent scarring tissue to form.

For example, a patient with decreased levels of a polypeptide receives a daily dose 0.1-100 ug/kg of the polypeptide for six consecutive days. Preferably, the polypeptide is in the secreted form.

The entire document of each document cited (including patents, patent applications, journal articles, abstracts or other disclosures) is hereby incorporated herein by reference. Further, the hard copy of the sequence listing submitted herewith and the corresponding computer readable form are both incorporated herein by reference in their entireties.

What is claimed is:

1. An isolated nucleic acid molecule comprising a nucleotide sequence selected from the group consisting of:
(a) the nucleotide sequence as set forth in SEQ ID NO: 3 (GAAAGGTGCCAATATTACAC);
(b) the nucleotide sequence as set forth in SEQ ID NO: 4 (GCTGGGTGTTTATGAAGTTG);
(c) a nucleotide sequence fully complimentary to (a) or (b).

2. A vector comprising the nucleic acid molecule of claim 2.

3. The vector of claim 2 further comprising heterologous promoter DNA.

4. The vector of claim 3 wherein the heterologous promoter DNA is operatively linked to SEQ ID NO: 3 or 4.

5. A host cell comprising the vector of claim 2.

6. The host cell of claim 5 that is a mammalian cell.

7. A liposome comprising the vector of claim 2.

8. The liposome of claim 7 wherein tumor homing molecules are present on the surface of the liposomes.

9. A vector for suppressing seprase mRNA translation comprising:
(a) a promoter operable in a mammalian host cell;
(b) an oligonucleotide sense sequence that targets a seprase mRNA gene sequence;
(c) an oligonucleotide spacer sequence; and
(d) an oligonucleotide anti-sense sequence of the same seprase mRNA gene sequence as in part (b), wherein the oligonucleotides from parts (b)-(d) form a short hairpin RNA that targets and forms complexes that destroy seprase mRNAs thereby preventing translation of seprase mRNA.

10. The vector of claim 9, wherein the promoter is a U6 promoter.

11. The vector of claim 9, wherein the oligonucleotide sense, spacer, and anti-sense sequence is set forth in SEQ ID NO: 3 or SEQ ID NO: 4.

12. The vector of claim 9, further comprising a transcription termination signal sequence downstream of oligonucleotide sequences of parts (b)-(d).

13. The vector of claim 12, further comprising a polynucleotide sequence encoding a detectable protein selected from the group consisting of AU1, AU5, FLAG, myc, HA, VSV-G, 6×His, green fluorescent protein, and yellow fluorescent protein.

14. A host cell comprising the vector of claim 13.

15. The host cell of claim 14 that is a mammalian cell.

16. A liposome comprising the vector of claim 13.

17. The liposome of claim 16 further comprises a tumor specific homing molecules on the surface of the liposome.

18. A method for inhibiting tumor intravasation by administering a therapeutically effective amount of a vector in a patient in need thereof wherein the vector comprises nucleotide sequences that reduce native seprase mRNA translation in tumor cells by forming short hairpin inhibitory RNAs that target and form complexes that destroy seprase mRNAs.

19. The method of claim 18 wherein the level of overall seprase activity is less in a tumor cell harboring the vector than in a tumor cell without the expressed short hairpin inhibitory RNA.

20. The method of claim 18, wherein the formation of hetero-oligometric protease complexes comprising seprase and dipeptidyl peptidases (DPP4/CD26) is prevented.

21. The method of claim 18, wherein the interaction between seprase and α3β1 integrin is prevented.

22. The method of claim 18, wherein the vector is harbored within a lipid based delivery system.

23. The method of claim 22, wherein the lipid based delivery system is a liposome.

24. The method of claim 23, wherein the liposome comprises homing molecules specific for tumor cells on the surface of the liposome.

25. The method of claim 18, wherein the tumor cells targeted are selected from the group consisting of: a melanoma cell, a breast cancer cell, a gastric carcinoma cell, a colonic carcinoma cell, and a cervical carcinoma cell.

26. The method of claim 18, wherein the vector comprises an oligonucleotide sequence as set forth in SEQ ID NOS: 3 or 4.

27. The method of claim 23 wherein the vector comprises an oligonucleotide sequence as set forth in SEQ ID NOS: 3 or 4.
